(19) United States
(12) Patent Application Publication
(10) Pub. No.: US 2016/0143594 A1
(43) Pub. Date: May 26, 2016

(54) MULTIDIMENSIONAL TIME SERIES ENTRAINMENT SYSTEM, METHOD AND COMPUTER READABLE MEDIUM

(71) Applicant: UNIVERSITY OF VIRGINIA PATENT FOUNDATION, Charlottesville, VA (US)

(72) Inventors: J. Randall MOORMAN, Keswick, VA (US); Douglas E. LAKE, Charlottesville, VA (US)

(21) Appl. No.: 14/900,448

(22) PCT Filed: Jun. 18, 2014
(86) PCT No.: PCT/US2014/043043

§ 371 Date: Dec. 21, 2015

Related U.S. Application Data
(60) Provisional application No. 61/837,508, filed on Jun. 20, 2013.

(51) Int. Cl. A61B 5/00; G06F 19/00

(52) U.S. Cl. CPC A61B 5/7275; A61B 5/742; A61B 5/7246; A61B 5/7257; A61B 5/726; A61B 5/4842; G06F 19/345

(57) ABSTRACT

Illness signatures are mathematically characterized by entrainment relationships among multiple time series representations of physiological processes. Such characteristics include time and phase lags, window lengths for optimum detection, which time series are most entrained with each other, the degree of entrainment relative to the rest of the large database, and the concordance or discordance of the time-varying changes. These optimum disease-specific characteristics can be determined, for example, from large, clinically well-annotated databases of time series representations of physiological processes during health and illness. These characteristics of the entrainment relationships among multiple time series representations of physiological processes are used to make mathematical and statistical predictive models using multivariable techniques such as, but not limited to, logistic regression, nearest-neighbor techniques, neural and Bayesian networks, principal and other component analysis, and others. These models are quantitative expressions that transform measured characteristics to the probability of an illness, or p(illness).

### CROSS-REFERENCE TO RELATED APPLICATIONS AND CLAIM OF PRIORITY

This application claims priority under 35 U.S.C. §119(e) and PCT Article 8, of copending U.S. Application Ser. No. 61/837,508 filed 20 Jun. 2013.

### FIELD OF THE INVENTION

The invention relates generally to a method, system, and computer readable medium for early detection of sub-acute potentially catastrophic illnesses, and more specifically to detecting abnormal entrainment of waveform and vital sign time series representations of physiological processes.

### BACKGROUND OF THE INVENTION

While patients in hospital beds and intensive care units differ in age, diagnosis, treatment, expected length of stay and prognosis, they all share one thing in common. They are all vulnerable to sub-acute, potentially catastrophic complications for which early diagnosis leading to early therapy should improve their outcomes. While early signs of impending problems may well be apparent to experienced clinicians, there are countless stories of sub-acute illness suspected too late. A general solution is to devise continuous monitoring algorithms that detect signatures of physiology going wrong. The need is for bedside monitoring that predicts sub-acute potentially catastrophic illness. Clinicians are challenged to make decisions based on current monitoring—only momentary displays of present values and limited, unwieldy views of trends. Doctors suspect, though, that better analysis of the multiple streams of data could detect subclinical deterioration. This would allow earlier diagnosis and therapy, and the promise of improved outcome. Experienced clinicians develop sixth senses about impending disaster, but would be hard-pressed to quantify their intuition or to be present at every bedside all the time.

Goldberger (1, 2), Buchman (3, 4) and others, who base their viewpoints of health and illness on concepts of non-linear dynamics, have suggested that time series representations of physiological processes contain complex information about how organs signal each other. In this context, the body is modeled as a collection of interconnected cells, organs and wiring incessantly adapting to circumstance through signals and responses. A widely observed manifestation is the variation in the times between heartbeats, a result of the highly responsive autonomic nervous system input to the sinus node. Thus heart rate variability (HRV) is a feature of healthy humans, and reduced HRV signifies illness. The interpretation is that illness leads to a reduction in complexity of human physiology, and to monotonous behavior that is oblivious to input signals. Thus, in this view, organs that ordinarily signal to each other are uncoupled during illness, especially by illnesses that lead to systemic inflammatory response syndrome such as sepsis and other infectious and non-infectious acute and chronic insults and injuries.

Multiple approaches exist within this framework. For example, HRV monitoring has been proposed, and is reported in 24 Holter monitor recordings and in implantable cardiac devices. An important variation of HRV analysis has been heart rate characteristics (HRC) analysis in premature infants. Here, the focus is on detection of a distinctly abnormal HR series with abnormal HRC of reduced variability and transient decelerations. A display that maps the degree of abnormal HRC to the fold-increase in probability of illness reduces mortality in neonatal ICUs.

These points of view, however, neglect the potentially important interactions among time series representations of physiological processes other than just the heart function. Frequency domain analysis of HRV gives indirect information on the interaction of the heart and lungs, as variation in HR in the frequency band of breathing, as determined by Fourier or Lomb analysis. The physiological basis of the analysis is that HR is modulated at different frequencies by the sympathetic and parasympathetic arms of the autonomic nervous system. Specifically, variation of HR in the frequency band that corresponds to the breathing rate is attributed to the vagus nerve and the parasympathetic nervous system. This is only indirect, as it does not require measurement of breathing. Nonetheless, this kind of analysis has been very widely studied, and inconsistencies exist. For example, breathing rate has profound effects on spectral analysis of high-frequency changes in HR that are unrelated to underlying physiology (5).

The work of Tracey and coworkers on the cholinergic anti-inflammatory pathway (6) show how infection and inflammation can cause changes in cardio-respiratory control. The finding is that there is vagal activation early in infection, and thus Tracey's model makes two predictions about, say, heart rate variability (HRV) and infection. First, activation of the vagus nerve early on should increase HRV (6). Indeed, studies in septic premature infants reveal prominent heart rate decelerations (7, 8). The vagus nerve has not been tested directly as the mechanism, as atropine can be dangerous in infants (9). In support of the idea, injection of microorganisms into adult mouse peritoneum leads promptly to heart rate decelerations that are clearly of vagal origin—there is AV block, and atropine promptly reverses the bradycardia (10). The second prediction is that chronically depressed vagal activity should predispose to infection. Indeed, many studies link reduced HRV to many chronic illnesses (11, 12). The response of HRV in the early stages of systemic infection was demonstrated by Seely and coworkers, who showed falling HRV over days prior to sepsis in patients who underwent bone marrow transplantation (13).

### SUMMARY OF THE INVENTION

An aspect of an embodiment of the invention includes methods, systems, techniques, computer readable media, and tools for detecting abnormal entrainment of multidimensional time series representations of physiological and disease processes.

Entrainment means that a physiological or disease process that has dynamical features—that is, it leads to time-varying changes in the patient's state—can lead to corresponding dynamical changes in physiological parameters. These include, but are not limited to, the commonly measured vital signs of heart rate, respiratory rate, and oxygen saturation of the blood that are available in all patients in ICU settings. These are examples of multidimensional time series representations of physiological and disease processes. A core idea is that small degrees of entrainment can be part of normal physiology, but that abnormally pronounced, high dimensional (involving more than 2 time series representations of physiological processes), prolonged, or otherwise unexpected degrees of entrainment represent illness.

The conceptual framework is that human physiology and pathophysiology are continuous time-varying processes that can be revealed by analysis of time series data measured by, for example, bedside EKG and hemodynamic monitors, or by personal monitors in the ambulatory setting, or by other means in other settings. Specifically, disease processes can entrain organ function and other aspects of physiological processes to the dynamical properties of the disease. A well-known example of such a dynamic illness is malaria, which leads to fever spikes at regular intervals. In fact, the periodicity of the fevers can be specific to the species causing the infection.

Thus, demonstrating dynamics of organ function can inform the clinician of changes in patient status, and can be especially useful in detecting early stages of illness when diagnosis and treatment can be most effective. Identifying and detecting patterns of abnormal entrainment can also lead to specific diagnoses, or indications or responses to specific therapies.

The current art consists of 1) measuring the mean and variability of individual time series of vital signs and other measured physiological parameters, 2) combination of them in multivariable statistical models, and 3) information about organ coupling, or how one organ influences another—a good example is respiratory sinus arrhythmia analyzed by frequency domain analysis of heart rate time series. These approaches lack the characteristic of detection of specific patterns of entrainment of time series representations of physiological processes. Rather, they describe general characteristics of multi-organ variability without analysis of joint time-variations that are pre-specified to be findings of illnesses.

The invention provides conceptual approaches and tools for detection of degrees of entrainment brought by illness. The invention is fundamentally different from measurements of means and variabilities of individual vital signs, such as heart rate (HR), heart rate variability (HRV), or the means and variabilities of other individually measured time series representations of physiological processes. The invention is applicable to all ages, as shown in the examples below.

The invention is fundamentally different from earlier concepts of organ coupling, or the synchronization of physiological processes that can accompany good health. It is well-known, for example, that states of calm relaxation lead to obvious synchronization of the heart and lungs. This phenomenon is well-recognized as the familiar respiratory sinus arrhythmia, and the mechanism is cyclical modulation of the activity of the vagus nerve, the action arm of the parasympathetic branch of the autonomic nervous system (14). It is well-known that states of calm lead to increased evidence of respiratory sinus arrhythmia, and that biofeedback and other techniques can modulate these phenomena. It is also well-known that illness reduces or abolishes respiratory sinus arrhythmia and other normal physiological entrainment phenomena. This concept of organ uncoupling has been demonstrated for sepsis and systemic inflammatory response syndrome, for example, and the mechanism is circulating endotoxin (15, 16).

This kind of normal entrainment of heart and lungs that is disrupted by illness is a counter-example to the core ideas of the invention. The new approaches described herein arise from the concept that a single time-varying process, namely, a dynamical disease, drives all aspects of time-varying physiological phenomena. Thus the appearance of related time-variations in one or more physiological processes can inform on the early stages of human illness. A unifying view that incorporates both this concept and the earlier concepts of normal organ coupling, entrainment and synchronization is that there are characteristic signatures of health and illness that can be detected in time series representations of physiological processes. Specifically, an example of healthy entrainment is respiratory sinus arrhythmia, and examples of illness-related entrainments are shown in the examples hereinbelow.

The various embodiments of the present invention (and aspects thereof) are fundamentally different from analysis of changes from baseline states. Rather, it detects abnormal patterns that are common to all patients, and not unique variations of an individual's repertoire of physiology. The various embodiments of the present invention (and aspects thereof) provide, among other things, insights and tools for analysis and interpretation of multidimensional time series representations of physiological processes that are available in monitored patients in intensive care units, emergency departments, hospital wards, operating rooms, outpatient centers such as for blood donation, chemotherapy infusions or hemodialysis, home visits, ambulances, and any other setting in which monitoring of physiological parameters can take place. Such a conceptual and analytical framework allows identification and detection of multidimensional pathophysiological signatures of illness.

One embodiment of the invention is a bedside display of the fold-increase in probability of an illness based on statistical analysis of the current multidimensional time series representations of physiological processes using predictive mathematical and statistical models. If results from multiple predictive models are available, the invention includes display of all of them or one or a few of them based on their properties as the largest, or the mean, or other.

### BRIEF DESCRIPTION OF THE DRAWINGS

FIG. 1 is a graph of a time series of HR, RR and O₂ saturation values;

FIG. 2 is a 30 minute plot from near hour 18 of FIG. 1, showing quasi-periodic fluctuations in the three signals, suggesting entrainment;

FIG. 3 is a 10-minute plot of FIG. 1, demonstrating a one-to-one correspondence of the fluctuations, most clearly in the RR and O2 saturation signals;

FIGS. 4A-4B, 5A-5B and 6A-6B are graphs showing pairwise correlations and coherences as a function of time for various time series of physiological processes;

FIG. 7 is a plot showing episodes of entrainment characterized by increases in HR and RR, and, later, simultaneous decreases in O2 saturation;

FIG. 8 is a 30-minute plot from near hour 16 of FIG. 7, showing the details of the entrainment, with simultaneous changes in the three time series representations of physiological processes;

FIG. 9 is a 10-minute scale plot, illustrating that when the time window is too short, details of the entrainment may be lost;

FIG. 10 is a graph of a time series of HR, RR and O₂ saturation values, showing respiratory decompensation prior to emergency unplanned intubation of the patient in the ICU;

FIG. 11 is a plot of FIG. 10 in hour 8 before emergency intubation;

FIG. 12 is another plot of FIG. 10 in hour 8 before emergency intubation;

FIGS. 13A-13B, 14A-14B, and 15A-15B are graphs showing correlations between entrainment of HR and RR in the patient of FIG. 10, leading to urgent unplanned intubation occurred at lag 0 to +10 seconds, and at frequency about 1 per minute;

FIG. 16 is a schematic outline of a study conducted in accordance with the invention;

FIG. 17 is a graph showing a rise in premature births in the US from 1990 to 2006;

FIG. 18 is an exemplary view of a bedside display of a predictive monitor in accordance with the invention;

FIG. 19 is a graph showing mortality reduction in NICUs using the monitoring system of the present invention versus conventional monitoring;

FIG. 20 is a predictiveness curve for neonatal HRC monitoring in large studies over a decade;

FIG. 21 is a plot showing NICU for 1100 infants in the University of Virginia Children's Hospital;

FIG. 22 is a heat map of respiratory support in 230 very low birth weight (VLBW) infants;

FIG. 23 is an event map of a VLBW infant who died of necrotizing enterocolitis (NEC);

FIG. 24 is a graph showing predictiveness curves for a monitoring system in the NICU in accordance with the invention, and a new SICU model;

FIG. 25 is a series of plots of univariate analysis of respiratory decompensation showing early detection of respiratory deterioration;

FIG. 26 is a plot of multivariate analyses of respiratory decompensation showing early detection of respiratory deterioration;

FIG. 27 is a graph showing the time course of model prediction for a 25-year-old man with rapidly increasing pleural effusion due to decompensated hepatic failure;

FIG. 28 is schematic diagram of a system architecture in accordance with an aspect of the invention;

FIG. 29 is a block diagram of a networked computer system usable with the present invention; and

FIG. 30 is a system in which one or more embodiments of the invention can be implemented using a network, or portions of a network or computers.

### DETAILED DESCRIPTION OF THE INVENTION

An aspect of an embodiment of the present invention may utilize (in whole or part) a large, clinically annotated database of multidimensional time series representations of physiological processes from which illness signatures can be deduced. An illness signature is a phenomenological description of alterations in multidimensional time series representations of physiological processes that are characteristic of early, subclinical phases of an illness. An example of an illness signature in a one-dimensional time series representation of a physiological process is the finding of abnormal heart rate characteristics of reduced variability and transient decelerations in early phases of neonatal sepsis. Examples of illness signatures in multidimensional time series representations of physiological processes are shown below.

Illness signatures are mathematically characterized by the entrainment relationships among multiple time series representations of physiological processes. Such characteristics include time and phase lags, window lengths for optimum detection, which time series are most entrained with each other, the degree of entrainment relative to the rest of the large database, and the concordance or discordance of the time-varying changes. These optimum disease-specific characteristics can be determined, for example, from large, clinically well-annotated databases of time series representations of physiological processes during health and illness.

These characteristics of the entrainment relationships among multiple time series representations of physiological processes are used to make mathematical and statistical predictive models using multivariable techniques such as, but not limited to, logistic regression, nearest-neighbor techniques, neural and Bayesian networks, principal and other component analysis, and others. These models are quantitative expressions that transform measured characteristics to the probability of an illness, or p(illness).

An aspect of an embodiment of the present invention includes global representations of multidimensional entrainment. For example, the global entrainment of commonly measured vital signs can be measured by summing the ranks of the pairwise cross-measures. In accordance with another aspect of the invention, the following general steps are performed:

optimally describe illness signatures using parameters of entrainment among time series representations of physiological processes in large clinically annotated databases;

for individual patients, mathematically characterize their m time series by m×n parameters selected by knowledge of illness signatures; and

calculate and display one or multiple p(illness) using predictive mathematical or statistical models that use the m×n parameters.

Specific representative steps in exercising the invention include:
Calculating mathematical characteristics of the relationships of one or more simultaneous time series in several domains using univariate measures and cross-measures including but not limited to the time-domain (e.g., autocorrelation, cross-correlation and covariance), frequency domain (e.g., frequency spectra using Fourier transform, Lomb periodogram or other techniques, cross-spectra, coherence, transfer functions), wavelet domain (e.g., cross-wavelet transform), non-linear domain (e.g., cross-entropy), phase domain (e.g., Hilbert transform), information domain (Granger causality and mutual information), and other mathematical and statistical domains using traditional or novel analyses, transforms, estimators or other mathematical calculations.
Determining cross-measures in specified ranges of time lags and frequency bands that are clinically and physiologically relevant and determined empirically from large databases—these are measures that are functions of two or more simultaneous time series representations of physiological processes. Entrained processes may operate with a phase shift manifesting as a time lag between time-varying features. That is, an increase in one measured parameter—heart rate, say—may be closely associated with a decrease in another measured parameter—oxygen saturation, say—at a later (or earlier) time. These classes of time lags that separate events in entrained series can be specific to disease processes. Their specification is empirical and based on large databases of time series collected during health and the early stages of illness. Concepts of cause and effect—“the oxygen saturation fell because the heart rate rose”—are not required. Part of the invention is specification of time and phase lags that are specific to time series representations of physiological processes, specific to kinds of illness, and specific to their proximity to clinical manifestations of illness.
   For example, we find that heart rate and oxygen saturation are entrained with an approximately 20-second lag within 12 hours of neonatal sepsis (Figures). Thus, for this example, the automated analysis of monitor data at the bedside comprises at least determination of the entrainment of heart rate and oxygen saturation using one or more cross-measures calculated at time lags near 20 seconds. Other disease processes might be optimally detected using different time lags. The Examples demonstrate the differences among patient types and among disease processes. The invention includes prescribed sets of time and phase lags and other parameter selections made from analysis of the large, clinically annotated database.
Restricting analyses to moving time windows of length appropriate for the dynamics of the disease. Optimum window lengths are developed for specific settings of diseases and data sets, and for the entrainment burden. The period or cycle length or average length of an epoch of entrainment may be determined empirically from large databases, without need for knowledge of physiological or pathophysiological processes. This is fundamentally different from multiscale approaches that are intended to capture, for example, transitions from sleep to waking, or from activity to inactivity. Rather, the time windows reflect the dynamics of the underlying disease process that entrains the physiological processes.
   Knowledge of an optimum window length allows interpretation of the results of entrainment analyses. Short window lengths are sensitive for detecting even transient epochs of entrainment. The appearance of entrainment in long windows, on the other hand, implies a high entrainment burden that is robust to averaging. Thus, for example, the automated analysis of monitor data at the bedside comprises at least determination of entrainment at multiple window lengths.
Calculating rates of change of measures and cross-measures using optimized window lengths, time and phase lags, and frequency bands. Entrainment may develop slowly or quickly, and rates of change of entrainment characteristics and parameters can be specific for individual diseases.
   It is well-known that the rates of onset of illnesses vary. Sepsis, for example, has a very different rate on onset—hours to a day or so—than acute myocardial infarction does—minutes or less. There is clinical information in the rate of change of entrainment, such that rapid rates might rule in or out illnesses in the differential diagnosis. Additionally, very stable and long-lived epochs of entrainment can signify fundamental, unchanging physiological states. For example, very prolonged and stable entrainment of HR, RR and O2 saturation in a newborn infant may signify immaturity of cardio-respiratory control, a risk factor of sudden infant death syndrome. Thus, for example, the automated analysis of monitor data at the bedside comprises at least determination of the rate of change of entrainment parameters.
Identifying the rank order of the cross-measures with regard to each other, i.e., identification of which time series and physiological processes are most related to each other to identify patterns of entrainments. Disease processes may specifically entrain some physiological processes more than others. Degrees of entrainment in health may vary among pairs or larger groups of time series representations of physiological processes, and patterns of entrained parameters can be specific for individual diseases. These relationships are determined empirically from large databases, without need for knowledge of physiological or pathophysiological processes. Thus, for example, the automated analysis of monitor data at the bedside comprises at least determination of the rank order of the specific entrainments with respect to each other.
Identifying the rank order of the cross-measures with regard to their expected distributions, i.e., identification of how extreme the measures and cross-measures are compared to a large database of observed values. The invention includes detection of extreme outliers of time series that are entrained. These relationships are determined empirically from large databases, without need for knowledge of physiological or pathophysiological processes. For example, the invention requires knowledge that the observed degree of entrainment is expected no more than a given percentage of the time, allowing assessment of the degree to which the value is an outlier. Comparing the measured value to a large database of similar measurements and determining the percentile in which the new observation falls can accomplish this. This obviates need for statistical demonstration of the normality of the distribution of the measured entrainments, as is required for, say, Z-score analysis, or counting the number of standard deviations away from the mean. Thus, for example, the automated analysis of monitor data at the bedside comprises at least determination of the rank order of the specific entrainments with respect to a large database of observed measures.
Determining the concordance or discordance among time series representations of physiological processes—that is, whether the entrainment leads to simultaneous or lagged joint increases and decreases (concordant), or to opposite changes in the values of one time series with respect to the other (discordant). The directionality of change among the entrained time series has clinical pathophysiological importance. Entrainment of time series representations of physiological processes can be concordant or discordant. An example of concordant entrainment is infants' response to sepsis or necrotizing enterocolitis or other illnesses, which are characterized by simultaneous falls in HR, RR and O2 saturation. An example of discordant entrainment is adults' response to respiratory decompensation leading to urgent unplanned intubation and other illnesses, which are characterized by simultaneous rises in HR and RR but falls in O2 saturation. Thus, for example, the automated analysis of monitor data at the bedside comprises at least determination of the concordance or discordance of specific entrainments.
Determining the percentile or rank of the observed measurement with respect to a large database.
Calculating p(illness) from multivariable statistical models that employ observed measures and/or their percentile or rank.
Displaying one or more selected p(illness) at the bedside.

It is noted that the entrainment of time series representations of physiological processes that signify early stages of illness may be periodic, may be at frequencies attributed to activity of the sympathetic or parasympathetic arms of the autonomic nervous system, and may be detectable using traditional Fourier- or Lomb-based, or novel frequency domain analysis. It is further noted that the entrainment may be manifested as linear, monotonic changes in the means of the time series representations of physiological processes, and may be detectable by linear regression. The entrainment also may have non-linear characteristics, and calculations in non-linear domains may be used.

Further, entrainment may be demonstrated or inferred from analysis of a single time series representation of physiological processes. For example, the reduced variability and transient decelerations of heart rate time series that occur early in the course of neonatal sepsis is viewed as entrainment of the heart rate by a disease process whose dynamics are reflected in control of the heart rate. This analysis of a single time series is an example of an illness signature arising from abnormal entrainment of time series representations of physiological processes during early, often sub-clinical stages of a significant human illness where early diagnosis and early treatment stand to improve health outcomes of individual patients. An example is the well-known phenomenon of reduced heart rate variability (HRV) during illness.

### Examples of Clinical Applications

### In Infants

### A. Late-Onset Sepsis

The NICHD Neonatal Research Network found a 2.5 fold increase in mortality and more than 30% increase in hospital stay in the 21% of VLBW infants with blood culture-proven late-onset sepsis (>3 days of age). Survivors of sepsis have a high risk of permanent neuro-developmental impairment. One strategy for improving outcomes is better methods for early detection of sepsis, through biomarker or physiomarker testing.

While it has long been recognized that heart rate time series in early stages have abnormal heart rate characteristics of reduced variability and transient decelerations, only recently has it been shown that some—but not all—of the decelerations are coincident with apnea (not breathing) episodes, and thus not necessarily associated with infection. Thus it is useful to characterize heart rate decelerations by whether or not there are simultaneous changes in the respiratory rate or oxygen saturation. A diagnostic aid might be to 1) detect decelerations—the current art—and to 2) report on the cross-correlation of heart rate and respiratory rate. If there is high correlation, this suggests decelerations due to apnea, and will lead the clinician to make a focused assessment of breathing. On the other hand, absence of correlation of the heart rate and the respiratory rate might lead the clinician to a focused assessment of infection.

### B. Necrotizing Enterocolitis

NEC occurs in up to 10% of very low birth weight (VLBW) infants, with an associated mortality up to 30% (17-19). An inflammatory response is central to the pathophysiology (20), and circulating cytokines are elevated (21). NEC survivors have a significantly higher risk of permanent neurodevelopmental impairment compared to age-matched controls, likely due to prolonged exposure to high levels of neurotoxic cytokines. Like in sepsis, earlier diagnosis of NEC might lead to earlier interventions that could be life-saving or brain-saving. Abnormal entrainment of time series representations of physiological processes may precede clinical diagnosis of NEC by several hours, allowing promise of earlier detection and life-saving therapy.

### C. Sudden Infant Death Syndrome

Sudden infant death syndrome (SIDS) is the most common cause of death in infants in the first year beyond the neonatal period (22). While the rate of SIDS has declined since 1997 coincident with the “back-to-sleep” campaign (23), there has been little change in the past decade and the rate remains significant, about 1 per 1700 live births (24). The etiology of SIDS is unknown, though it is thought to be related to improper neurological development of control centers for arousal, breathing and heart rate in the brainstem (25, 26). Abnormal entrainment of time series representations of physiological processes in the newborn period can reflect immaturity of cardiorespiratory control, and can identify newborns at higher risk of sudden infant death syndrome.

### In Adults

### A. Sepsis

Sepsis is a bacterial infection of the bloodstream, that is common in ICU patients and has a >25% risk of death. In 2006, Shannon and coworkers estimated the cost of a central line associated bloodstream infection (CLABSI) to be more than $26,000 (27). Martin and coworkers reported a yearly increase of nearly 10% in the US from 1979 to 2000, about three-fold over two decades, and last seen at 660,000 cases in 2000 (28). The yearly costs exceed $17B. Since some cases that develop during hospitalization are the result of, for example, central venous catheters, CMMS has declined reimbursements costs and charges for them, lending urgency to better, earlier detection.

### B. Respiratory Decompensation

Respiratory decompensation leads to urgent, unplanned intubation, which results in increases in length of stay and mortality of the patient. In addition to the personal discomfort of mechanical ventilation, there is the risk of ventilator-associated pneumonia, a diagnosis with high morbidity and mortality. Better detection of early phases of respiratory decompensation may lead to prompt trials of bronchodilators, supplemental oxygen, or more aggressive though still non-invasive ventilatory modalities and thus to avoidance of intubation altogether.

### C. Congestive Heart Failure

Congestive heart failure of any cause should have early phases where treatment might improve outcomes. The use of Swan-Ganz catheterization to make the early diagnosis of volume overload has been controversial, with either no impact on outcome (29) or a negative one (30). Clearly, a non-invasive method to detect volume overload and the need for diuresis would be very useful.

### Examples of Multidimensional Entrainment of Time Series Representations of Physiological Processes

In early phases of neonatal necrotizing enterocolitis (NEC), which, like clinical or proven neonatal sepsis, is a systemic inflammatory response syndrome, there are prolonged and pronounced entrainment of the heart rate, respiratory rate and oxygen saturation, the three vital sign time series currently available from bedside monitors. Entrainment can also occur in infants destined for sudden infant death syndrome (SIDS).

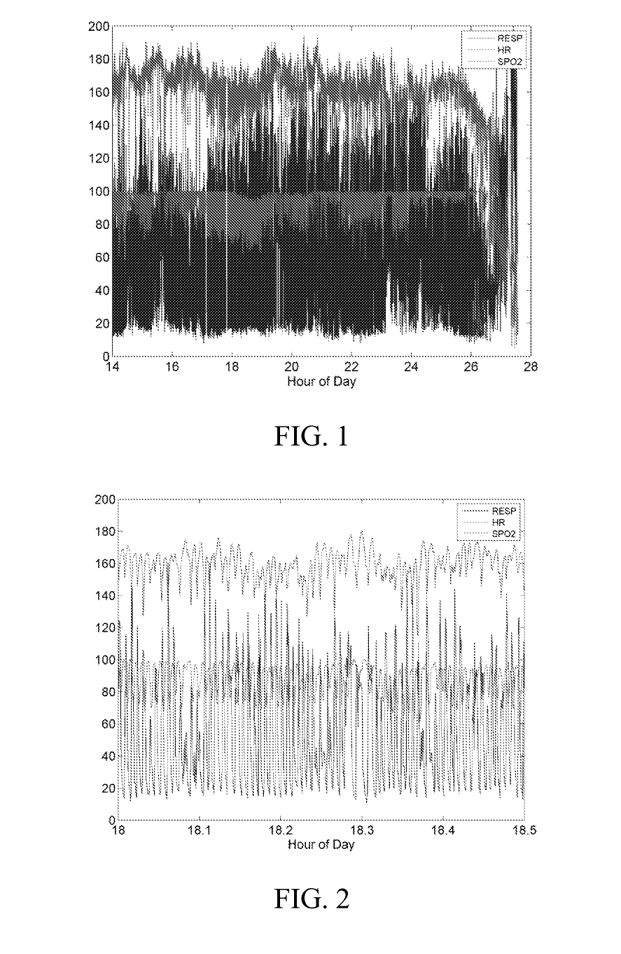

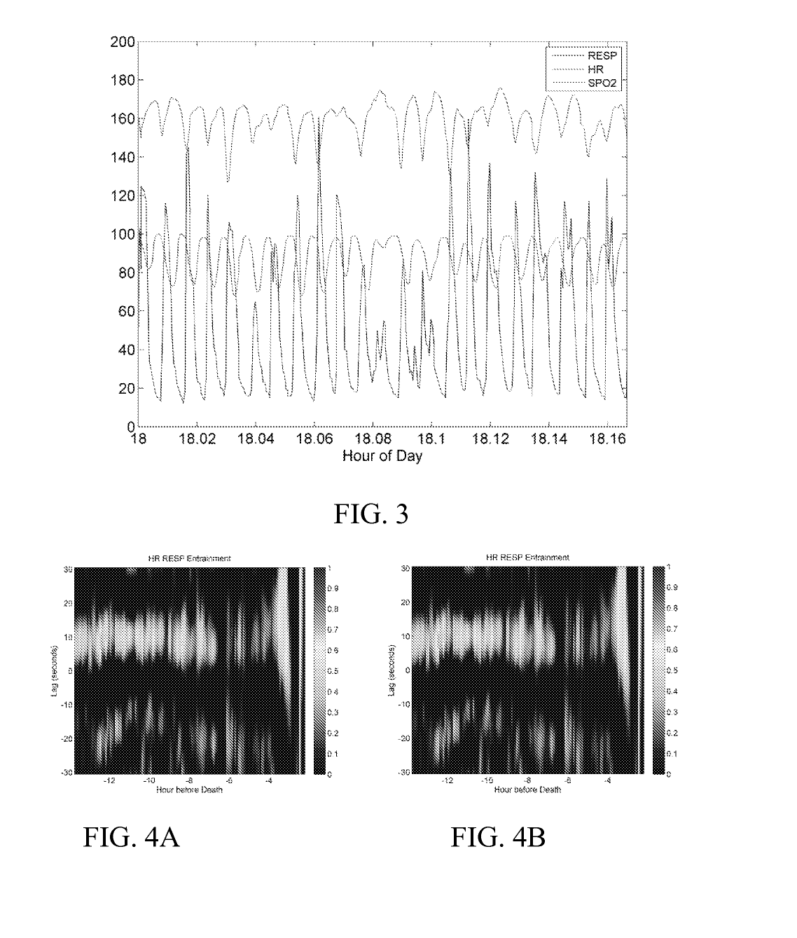

FIG. 1 shows plots of 3 readily available vital signs—the heart rate (green), respiratory rate (blue), and oxygen saturation measured from plethysmography (red). These are non-invasively measured from skin electrodes and a pulse oximeter. FIG. 1 shows recordings from the 14 hours prior to sudden unexpected death of a premature infant in the Neonatal Intensive Care Unit. Specifically, FIG. 1 shows 14-hour time series of HR, RR and O₂ saturation in an infant who died near hour 28 of suspected fulminant late-onset neonatal sepsis. At this scale, the abnormalities are high variability of the O₂ saturation (red) and RR (blue), but details are not evident because the time scale is too long. FIG. 2 shows a 30 minute plot from near hour 18 shows quasi-periodic fluctuations in the three signals, suggesting entrainment. FIG. 3 shows a 10-minute plot that demonstrates a one-to-one correspondence of the fluctuations, most clearly in the RR and O₂ saturation signals. The frequency is about 2 per minute.

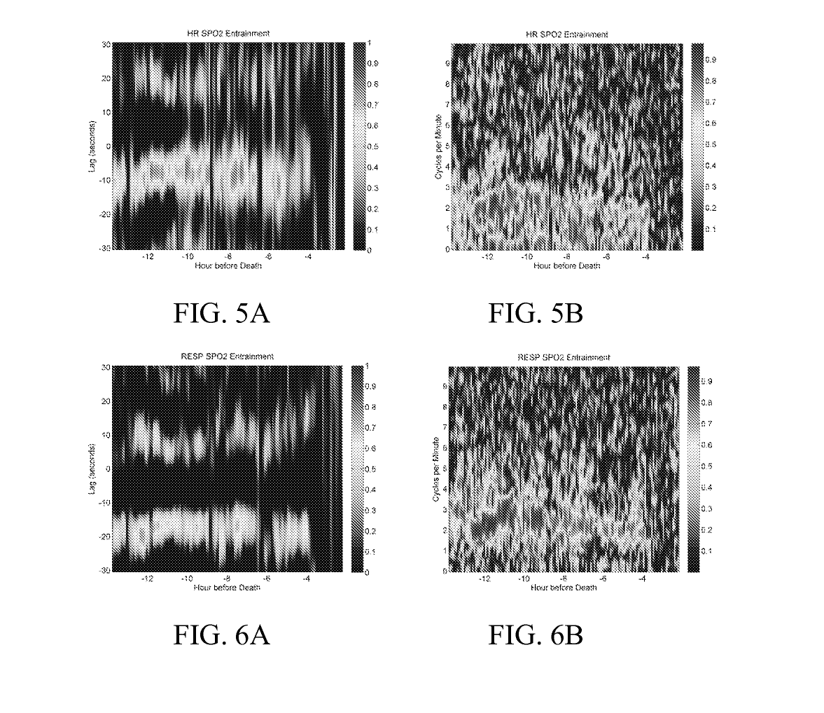

The second case, FIGS. 4A-6B, shows recordings from the same period of time prior to respiratory decompensation leading to urgent unplanned intubation in an adult in the Surgery/Trauma/Burn ICU. The findings are of entrainment of the time series representations of physiological processes. There are important differences in the time scales, phase lags, and optimum time window for analysis.

FIGS. 4A-6B show pairwise correlations and coherences as a function of time, with color scales to the right. The lags differ—+5 to +15 seconds for HR and RR, −10 to 0 seconds for HR and O₂ saturation, and −20 to −10 seconds for RR and O₂ saturation. For each, the coherence resides at a frequency near 2 per minute. Note the very prolonged duration of the entrainments—most of the 14 hour period displayed leading up to death.

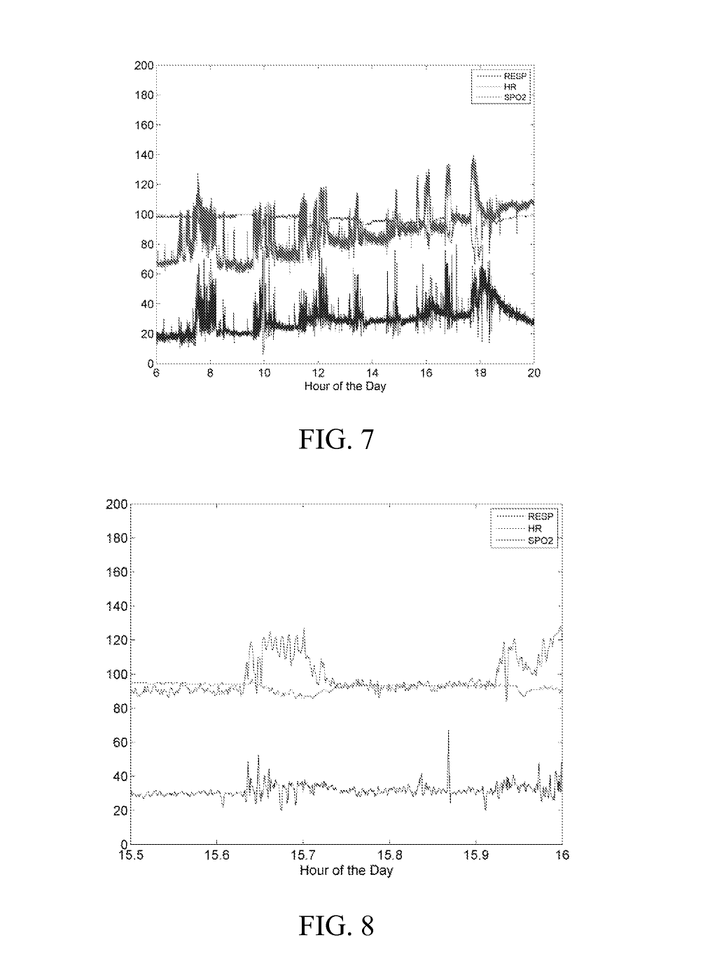

FIG. 7 shows 14-hour plots illustrating episodes of entrainment characterized by increases in HR and RR, and, later, simultaneous decreases in O₂ saturation. The episodes are clearly distinct at this time scale, and have frequency about 1 per hour.

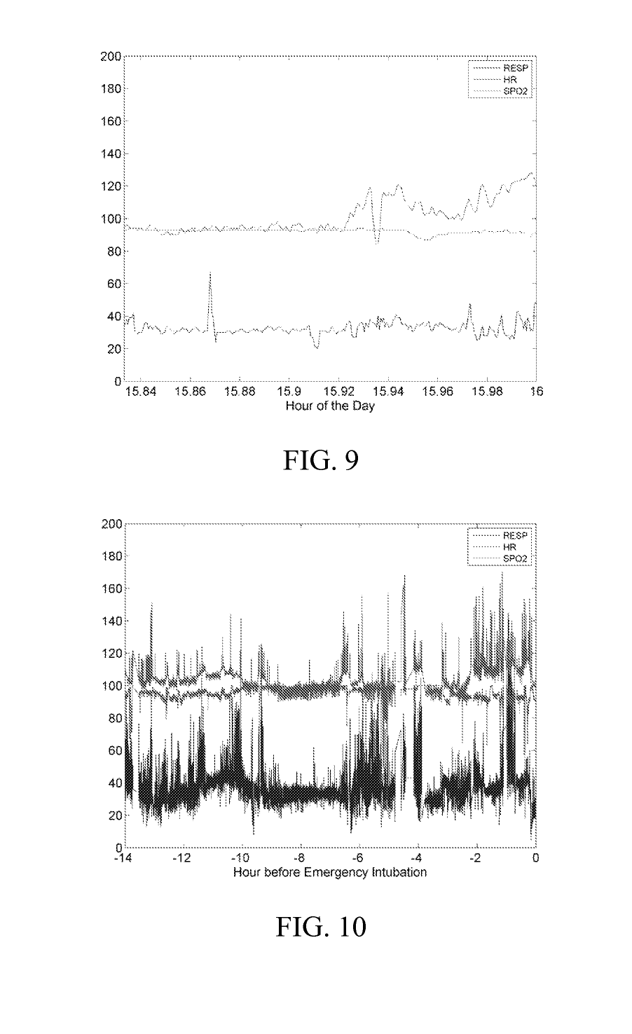

FIG. 8 shows a 30-minute plot from near hour 16 of FIG. 7, and illustrates the details of the entrainment, with simultaneous changes in the three time series representations of physiological processes. FIG. 9 shows a 10-minute scale, illustrating that details of the entrainment are lost when the time window is too short.

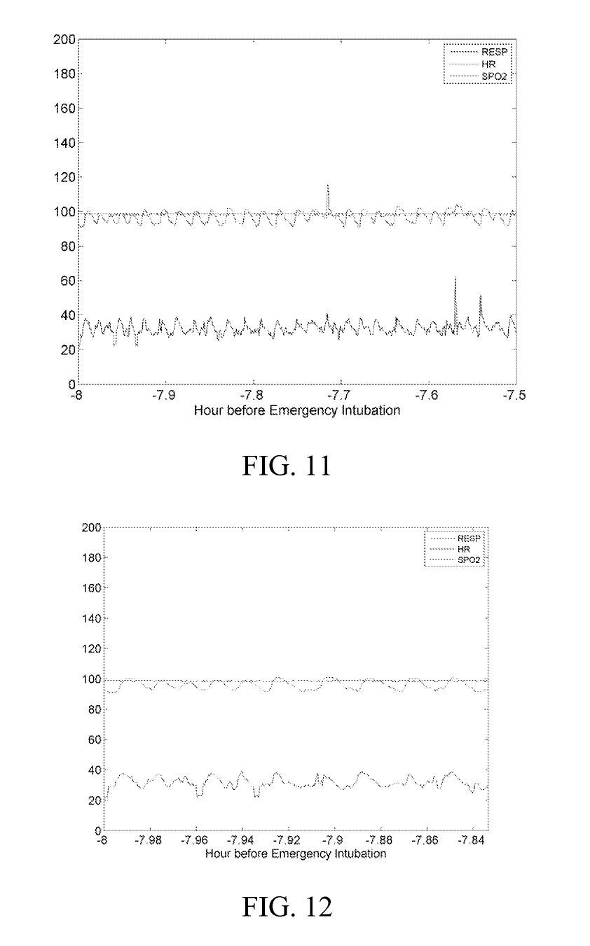

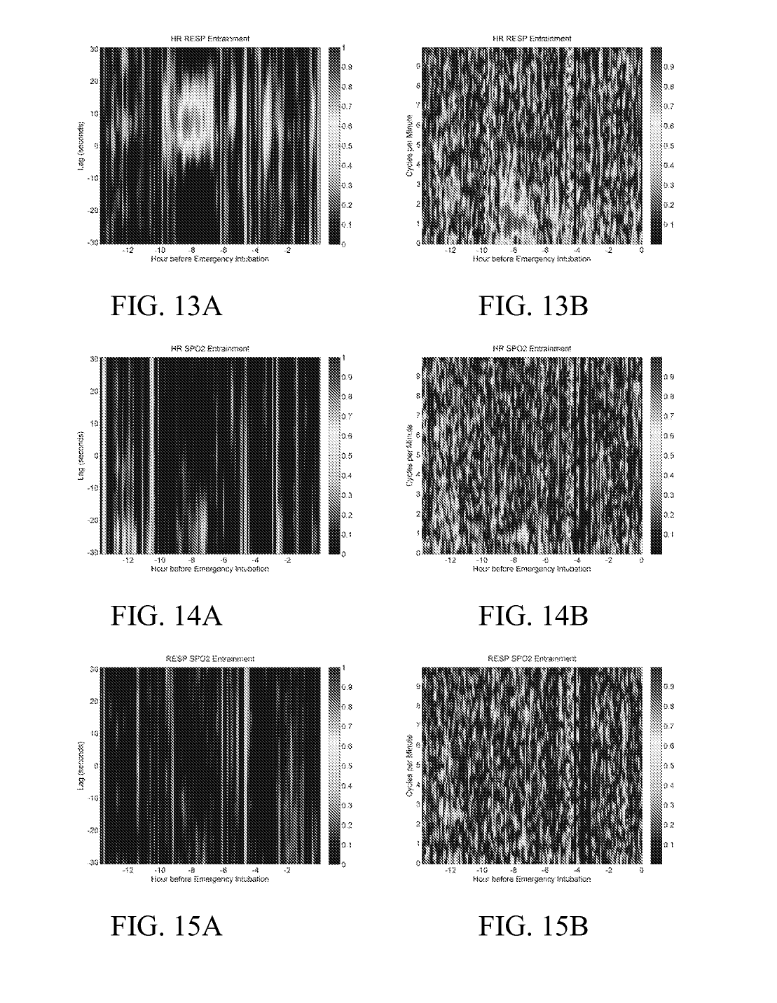

FIGS. 10-15B are analogous to FIGS. 4A-6B and 7-9, and illustrate a third example of respiratory decompensation leading to urgent unplanned intubation in the Medical ICU. The entrainment of HR and RR in this adult with respiratory decompensation leading to urgent unplanned intubation occurred at lag 0 to +10 seconds, and at frequency about 1 per minute. The entrainment was brief—about an hour—and was manifest only in the HR and RR analysis.

### Predictive Monitoring in Intensive Care Units

While patients in intensive care units throughout a modern tertiary care hospital differ in age, diagnosis, treatment, expected length of stay and prognosis, they all share one thing in common. They are all vulnerable to sub-acute, potentially catastrophic complications for which early diagnosis leading to early therapy should improve their outcomes. While early signs of impending problems may well be apparent to experienced clinicians, there are countless stories of sub-acute illness suspected too late. A general solution is to devise continuous monitoring algorithms that detect signatures of physiology going wrong.

This idea stands on the work of Goldberger, Buchman and others, who based their viewpoints of health and illness on concepts of non-linear dynamics. In this context, the body is a collection of interconnected cells, organs and wiring incessantly adapting to circumstance through signals and responses. A widely observed manifestation is the variation in the times between heartbeats, a result of the highly responsive autonomic nervous system input to the sinus node. Thus heart rate variability (HRV) is a feature of healthy humans, and reduced HRV signifies illness. It is clear that there are many appearances of normal heart rate time series, but only one during illness. The interpretation is that illness leads to a reduction in complexity of human physiology, and to monotonous behavior that is oblivious to input signals. These are powerful concepts not previously harnessed and reduced to the practice of medicine.

We have pioneered the bedside application of these ideas. We began our work in the Neonatal ICU with the goal of early detection of neonatal sepsis. This is a clear example of the kind of illness where early detection and early therapy with antibiotics should favorably alter the course of the illness. We found a signature of pathophysiologic dynamics in the heart beat intervals, and we developed a predictive model based on detection of the abnormal heart rate characteristics (HRC) of reduced variability and transient decelerations using, among other things, tools of non-linear dynamical analysis. In a very large randomized trial, we made the remarkable finding of improved survival in HRC-monitored infants.

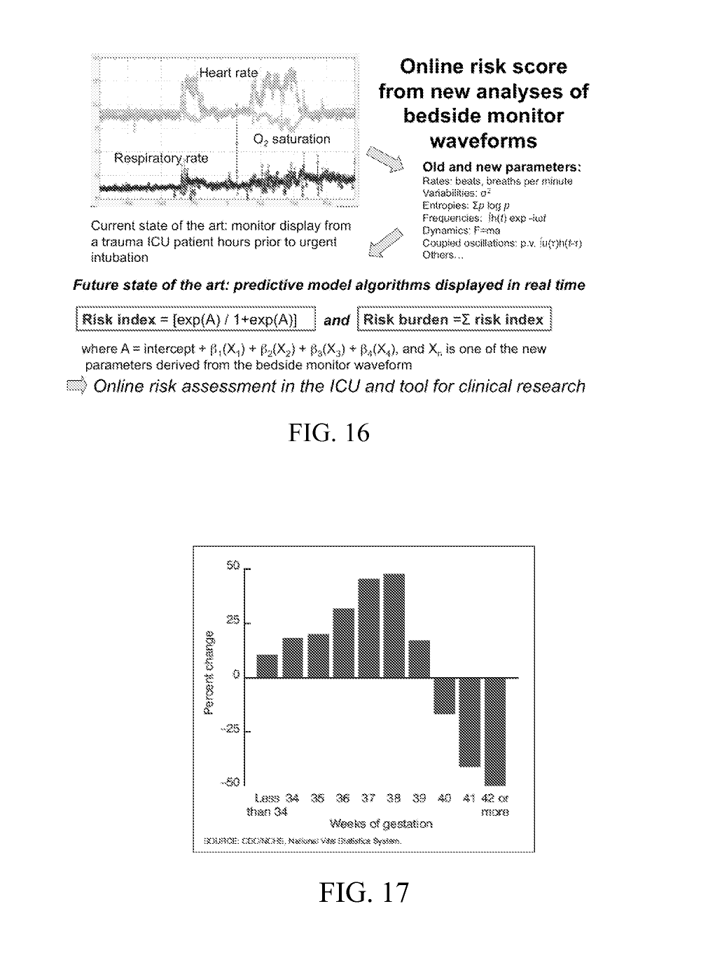

The present invention now extends our methods of discovery, development and clinical trials to other intensive care units—the Pediatric ICU, Surgical, Trauma & Burn ICU, Medical ICU, Coronary Care Unit, and Neurological ICU. In each, the focus is on the major clinical scenarios for which early diagnosis should improve outcome—the recurring themes are sepsis, urgent intubation, bleeding, and worsening heart failure. We will also apply these methods to monitored ward patients with the goal of informing rapid response teams. FIG. 16 provides an overview of our work.

### Challenge, Innovation and Impact Statement

We intend to change the way that medicine is practiced in hospitals through bedside monitoring that predicts sub-acute potentially catastrophic illness. Clinicians are challenged to make decisions based on current monitoring—only momentary displays of present values and limited, unwieldy views of trends. Doctors suspect, though, that better analysis of the multiple streams of data could detect subclinical deterioration. This would allow earlier diagnosis and therapy, and the promise of improved outcome. Experienced clinicians develop sixth senses about impending disaster, but would be hard-pressed to quantify their intuition or to be present at every bedside all the time.

We envision continuous monitoring that detects physiology going wrong. This requires new alliances between expert clinicians and quantitative scientists, and large-scale computing optimized for testing novel algorithms in very large data sets with meticulous clinical annotation. Our innovations are (1) highly granular clinical thinking by a critical mass of clinicians arm in arm with (2) wide-ranging mathematical thinking by quantitative scientists, and (3) proof of principle—our team has developed predictive monitoring that saves the lives of premature infants in the Neonatal ICU (31). The potential impact of this work is a 20 to 30% reduction in all ICU mortality.

### Rationale

We wish to save lives of patients admitted to ICUs. Their mortality is high enough based simply on the severity of the original injury or illness, but is further raised by events during their stay. We target those that are sub-acute but potentially catastrophic, such as infection. Sepsis, for example, is a bacterial infection of the bloodstream, that is common in ICU patients and has a >25% risk of death. Logically, early detection and treatment with antibiotics should improve outcomes. Our fundamental precepts are:
some potentially catastrophic medical and surgical illnesses have subclinical phases during which early diagnosis and treatment might have life-saving effects
these phases are characterized by changes in the normal highly complex but highly adaptive regulation and interaction of the nervous system and other organs such as the heart and lungs
teams of clinicians and quantitative scientists can work together to identify clinically important abnormalities of monitoring data, to develop algorithms that match the clinicians' eye in detecting abnormalities, and to undertake the clinical trials to test their impact on outcomes

We are buoyed by our recent proof of these concepts. We studied such an illness (late-onset sepsis in premature infants) with a subclinical abnormality of physiologic regulation (reduced heart rate variability punctuated by transient decelerations) developed algorithms to detect these abnormal heart rate characteristics (HRC), showed a relevant relationship of an HRC index to early diagnosis, and performed a very large randomized clinical trial to test its impact on neonatal outcomes. The trial was very simple—infants were randomized to have the HRC index shown to health care personnel, or not to be shown. The trial had a most important finding—a more than 20% reduction in mortality for HRC-monitored infants.

The critical barriers to progress have been the lack of high speed computing, the lack of novel quantitative thinking, and, probably most importantly, the lack of communication between the clinical and quantitative worlds. The state of the art remains threshold-based logic implemented on bedside monitors. Clinicians are shown events meeting bright cut-off alarm criteria that are often merely artifact, and there is no integrated assessment of multiple data streams. The computing barrier has been surpassed, so the needed data set can now be collected. The challenge of new quantitative thinking, however, has been unmet prior to the present invention.

The application begins with an overview of the entire project followed by a short account of the neonatal sepsis work and a recent clinical trial. We then outline our organizational structure, emphasizing the collaboration of clinicians with quantitative scientists and the existing hardware and software, and our clinical and mathematical methods. We finish with a quick tour of the ICUs and their opportunities, thoughts on our fit with the TR01 mechanism, and a timeline.

### Significance

The number of premature births is rising (32), and so are NICU admissions for the complex care of VLBW infants (33). FIG. 17 shows the striking rise in premature births between 1990 and 2006. The course of post-natal development of the VLBW infant in the NICU centers on support of ventilation and nutrition while systems mature. There are, however, interruptions by the apparently sudden onset of inflammatory illnesses such as sepsis and necrotizing enterocolitis. The mortality is high—for sepsis with gram-negative organisms, it can exceed 50%—and there is substantial short- and long-term morbidity (34, 35). These illnesses are not really sudden, though—clinical signs of illness occur relatively late in the course, when the systemic inflammatory response is well-developed. What has been lacking is an effective system of early detection that allows early treatment.

Many have postulated that new analysis of the existing data from the standard-of-care bedside monitors should give clues that are useful in early diagnosis. The UVa group has reduced one such analysis to clinical practice. They found abnormal heart rate characteristics (HRC) of reduced variability and transient decelerations in the heart rates of septic infants for 12 to 24 hours and more prior to clinical suspicion. They developed mathematical tools to detect abnormal HRC, validated them externally, demonstrated their relationship to clinical data such as lab results and other findings, and performed a large randomized trial to assess impact on VLBW outcome. The results were important—a more than 20% mortality reduction when HRC monitors were displayed—and no other intervention mandated—to clinicians (31). These findings are detailed further below in Preliminary Results.

This is only one target illness, and only one physiological signal. We hypothesize that other illnesses also have subclinical prodromes to target for early diagnosis, and that the heart rate is not the only source of information. The framework for mechanistic thinking is that the developing capacity for cardio-respiratory control is derailed by systemic inflammation, leading to altered heart and respiratory rates, patterns and couplings. Frameworks that integrate clinical and molecular ideas are those of the systemic inflammatory response syndrome proposed by Bone (36), and the cholinergic anti-inflammatory pathway proposed by Tracey (6). The point of view is that quantifying the control of heart and breathing rate, and their interaction, will lead to life-saving clinical monitoring. In this regard, the work of Tracey has been explicit on the involvement of the vagus nerve in the response to infection. As a result, the focus of this work is on heart rate characteristics, apnea, and respiratory sinus arrhythmia, a phenomenon that requires strong coupling of heart and lung, never a feature of severe illness.

The critical barriers to progress have been the lack of high speed computing, and the lack of novel quantitative thinking. The state of the art remains threshold-based logic implemented on bedside monitors. Clinicians are shown events meeting sharp cut-off criteria that may be mere artifact, and there is no integrated assessment. The computing barrier has been surpassed. The challenge of new quantitative thinking, however, is largely unmet. The group can meet this challenge with its multidisciplinary team of clinicians and applied mathematicians, and can apply the lessons learned in developing heart rate characteristics (HRC) monitoring.

There is in particular great value in improving the outcomes of VLBW infants. In addition to the human reward of saving the lives of tiny babies, there is a financial incentive. With a daily NICU cost of $1200 for the approximately 5% of the 4.2 million babies born annually in the U.S., reducing NICU length of stay by only two days could save over $0.5B yearly. The future goals include randomized trials to test the impact of this new monitoring on neonatal outcome.

The work centers on the relationship between cardiorespiratory control and neonatal illness.

### 1. Cardiorespiratory Control

### A. Control of Neonatal Breathing: Central Neonatal Apnea, or Apnea of Prematurity (AOP)

AOP is a pause in respiration >20 seconds, or <20 seconds if accompanied by bradycardia or O₂ desaturation. It is found in >50% of infants with birth weight <1500 g and in virtually all infants born <1000 g (37). The gold standard for documenting apnea, a polysomnograph, is impractical in the NICU. The default measure, NICU nurses' written documentation, has long be known to be unreliable in reporting the true occurrence (38). Uncertainty about AOP prolongs NICU stay for many preterm infants (39, 40). While AOP typically resolves between 35-37 weeks postmenstrual age (PMA), many preterm infants continue to have physiological immaturity, including apnea, bradycardia, and desaturation events, until well beyond 40 weeks (41). AOP persists at later PMA in those born at earlier gestations (42, 43), reflected in the large increase in length of stay for them—the unchanging mean of 80 days stay for infants with birth weight 751 to 1000 g is about twice that of infants with birth weight 1251 to 1500 (39). Due to concern that recurrent AOP at home could lead to death or rehospitalization, the practice is to delay NICU discharge until some time after the last apnea episode that is not otherwise explained—in our hospital, this means 8 apnea-free days (40).

### B. Control of Neonatal Heart Rate: Abnormal Heart Rate Characteristics Prior to Neonatal Sepsis

The work of the group has been to characterize the phenomenon of reduced variability and transient decelerations prior to clinical neonatal illness (7, 8, 10, 31, 44-59). While the mechanism is not known, the group's most recent translational experimental work suggests that cytokines released as part of the inflammatory response to infection may alter HRC through a vagal mechanism (10) as predicted by the cholinergic anti-inflammatory pathway of Tracey (6).

### 2. Sub-Acute, Potentially Catastrophic Neonatal Illness

### A. Late-Onset Neonatal Sepsis

Infants in the NICU are at risk for sepsis, and those at highest risk are those born with very low birth weight. Approximately 56,000 VLBW infants are born in the United States each year (60). Survival of this group has improved with advances in neonatal intensive care, but late-onset sepsis continues to be a major cause of morbidity and mortality (35). In a large study, the NICHD Neonatal Research Network found a 2.5 fold increase in mortality and more than 30% increase in hospital stay in the 21% of very low birth weight (<1500 g, VLBW) infants with culture-proven sepsis. Sepsis was the leading cause of NICU death after the first week of life, accounting for 45% of late deaths, half of which were sudden and unanticipated (61). These findings led the NICHD NRN them to conclude that strategies to reduce the incidence and severity of neonatal sepsis are “needed urgently”. In our RCT, the rate of sepsis was 25%. HRC monitoring in this subgroup reduced mortality from 16% to 10%.

### B. Necrotizing Enterocolitis

This poorly understood, multifactorial illness occurs in up to 10% of premature infants, and has mortality up to 30% (17-19). An inflammatory response is central to the pathophysiology (20), and circulating cytokines are elevated (21). Like sepsis, early diagnosis might lead to life-saving intervention.

### C. Intracranial Hemorrhage

Intracranial hemorrhage is more likely in infants with altered heart rate dynamics (62). Our group found that cumulative HRC measure is associated with brain injury and neurodevelopmental outcome (63). DRIFT (drainage, irrigation and fibrinolytic therapy) has potential to decrease morbidity (64). Earlier identification of IVH could allow an immediate brain ultrasound could be performed to see if an IVH had occurred and if so, DRIFT applied. Furthermore, early detection of a small germinal matrix hemorrhage might well lead the clinician to institute clinical interventions, such as ventilator, fluid, coagulation, and/or sedation adjustments, that might prevent extension of the hemorrhage.

Patients admitted to ICUs face high enough mortality based on the severity of the original illness, but the mortality risks are made even higher by new events that are not diagnosed until late in their course.

1. Worsening congestive heart failure of any cause should have early phases where treatment might improve outcomes. In the CCU, patients have complex, severe heart disease, and are vulnerable to volume overload along with ischemic events, arrhythmias, and a host of non-cardiac ills. Early detection of volume overload that allows early intervention should improve management and lead to shorter CCU stays. In ambulatory patients with cardiac implanted electronic devices (CIEDs) early detection of reduced heart rate variability (HRV) (65), decreasing transthoracic impedance (66) or increasing left atrial pressure (67) improve management. The use of Swan-Ganz catheterization to make the early diagnosis of volume overload has been controversial, with either no impact on outcome (29) or a negative one (30).

2. Respiratory decompensation for pulmonary as well as cardiac reasons leading to urgent, unplanned intubation results in increases in length of stay and mortality. In addition to the personal discomfort of mechanical ventilation, there is the risk of ventilator-associated pneumonia, a diagnosis with high morbidity and mortality. Better detection of early phases of respiratory decompensation may lead to prompt trials of bronchodilators, supplemental oxygen, or more aggressive though still non-invasive ventilatory modalities and thus to avoidance of intubation altogether.

3. Sepsis is a bacterial infection of the bloodstream, that is common in all ICU patients, including the CCU, and has a >25% risk of death. There were yearly increases of nearly 10% from 1979 to 2000, about three-fold over two decades, and last seen at 660,000 cases in 2000 (28). The cost of a central line associated bloodstream infection (CLABSI) to be more than $26,000 (27). The yearly costs exceed $17B. Since some cases that develop during hospitalization are the result of central venous catheters, CMMS has declined reimbursements costs and charges for them, lending urgency to better, earlier detection.

These illnesses share systemic inflammation as part of the pathophysiology. For example, heart failure has an inflammatory footprint (68), and a current view is that unchecked cytokine production mediated by NK-kappaB promotes apoptosis and adverse cardiac remodeling (69, 70). We look to the work of Tracey and coworkers on the cholinergic anti-inflammatory pathway (6) to conceive how inflammation alters cardiorespiratory control. The basic finding is of vagal activation early in inflammation, and thus the model makes two predictions about heart rate variability (HRV) in this setting. First, activation of the vagus nerve early on should increase HRV (6). Indeed, our studies in septic premature infants reveal prominent heart rate decelerations (7, 8). The vagus nerve has not been tested directly as the mechanism, as atropine can be dangerous in infants (9). We have found, though, that injection of microorganisms into mouse peritoneum leads promptly to heart rate decelerations that are clearly of vagal origin—there is AV block, and atropine promptly reverses the bradycardias (10). The second prediction is that chronically depressed vagal activity should predispose to inflammation. Indeed, many studies link reduced HRV to many chronic illnesses (11, 12), especially heart failure.

Thus our fundamental precepts are:
some CCU illnesses have subclinical phases when early diagnosis and treatment might save lives;
these phases are characterized by changes in vagally-mediated cardiorespiratory control;
clinicians and quantitative scientists can work together to develop algorithms that match the clinicians' eye in detecting abnormalities, and to do clinical trials to test their impact on outcomes.

The critical barriers to progress have been the lack of high speed computing, the lack of novel quantitative thinking, and, probably most importantly, the lack of communication between the clinical and quantitative worlds. The computing barrier has been surpassed, so the needed data set can now be collected. Our group is rising to the other challenges, and has met with success in studies of HRC monitoring in infants.

Early detection of inflammation-mediated changes in cardio-respiratory control can help solve the problem of ICU mortality be allowing earlier diagnosis and therapy.

### Precepts

Monitoring data, formerly evanescent, hold much information about health, illness, and about the response of cardio-respiratory control to inflammatory illness.
The combined efforts of clinicians, mathematicians and engineers can lead to effective new monitoring strategies that allow early diagnosis of otherwise invisible clinical deterioration.
Improved analysis of routine bedside monitoring data will favorably impact patient outcomes.

### Study Design

This is a 5 year project to assemble a large and novel database of physiological monitoring, to develop new analytical metrics, and to develop and internally validate predictive and diagnostic monitoring tools. A review of the UVa NICU for the past 5 years projects we can anticipate 7500 patient days from 125 VLBW patients in the NICU yearly. The database holds more than 300 VLBW infants now, and the number will rise to about 400 at the beginning of the proposed study period. Adding no less than 600 yearly for the study period leads to the estimate of 1000 VLBW infants characterized. We propose to:
Calculate the frequency, duration and burden of apnea using our new automated detector
Calculate the degree and duration of respiratory sinus arrhythmia (RSA), especially phase-locking of heartbeats and breaths, after optimizing our new breath-by-breath RSA detector
Characterize, categorize, and catalogue all events of sepsis, necrotizing enterocolitis and intracranial hemorrhage—this will call for much expert clinical consideration and judgment
Similarly, characterize, categorize, and catalogue all information relevant to apnea such as respiratory support and nursing entries
Record these clinical data in a SQL database connected to the waveforms and vital sign database
Inspect physiologic waveform data to determine characteristics of early stages of illness
Develop new or optimally adapted mathematical measures that report on abnormal waveforms and other relevant characteristics, starting with our breath-by-breath RSA and apnea detectors
Construct predictive models from the qualitative and quantitative analyses using multivariable techniques such as (but not limited to) logistic regression and nearest-neighbor techniques
Use data from the first 2.5 years to develop algorithms and from the second 2.5 years to validate them internally

In the mission to develop novel monitoring for early detection of sub-acute potentially catastrophic illness, our approach is:

### 1. Pick the Right Problem

This is the hardest part. We seek clinical scenarios in which there is a subclinical phase where we might expect that early diagnosis and treatment will improve outcomes. Neonatal sepsis is the perfect example—common (25% of very low birth-weight infants), deadly (mortality 50% higher, about 20% overall), and no good clinical signs to alert the clinicians. On the other hand, ventricular tachyarrhythmia in adults with heart disease is not as good a target. While common, deadly and without early detection strategies, there is no immediately preventive measure. Implanted defibrillators, which await the problem but then rapidly treat it, will be hard to surpass.

### 2. Look at the Data

This is the most time-consuming part. Clinicians and mathematicians spend hours together looking at the physiological waveform and vital sign records for patients who had the events listed above. We identify with our eyes the features that we wish to quantify—for example, this is how we found reduced variability and transient decelerations prior to neonatal sepsis.

### 3. Fear No Math

This is the most interesting and fun part. We do not subscribe blindly to the idea that physiological variability and its frequency components hold all the answers, though we agree the idea of reduced complexity during illness is a very useful framework. Reduced variability and transient decelerations would never have come to light, though, had we used only traditional heart rate variability measures because they do not detect them when they occur together. Here is the work of the quantitative scientists, then, to reduce the observations of the clinicians to measured parameters, often novel.

### 4. Do Clinical Trials

This is the most nerve-wracking part. We developed heart rate characteristics analysis in 4 years, and then spent nearly 7 in a randomized trial that is described below. The outcome was emphatic—more than 20% mortality reduction in monitored infants. But there were some surprises—no increase in antibiotic use, for example, though we had expected the monitoring to lead to more courses of sepsis treatment. Randomized trials are the only way to convince clinicians to change their practice, and this is how it should be—too many clinical practices have vaporized in the face of randomized trials, such as the use of anti-arrhythmic drugs to prevent sudden cardiac death, or hormone replacement after menopause to prevent heart disease. We anticipate that predictive monitoring, though, will improve outcomes and not lead to new harms, at least based on the trial we describe below.

This is an innovative approach:
the simultaneous evaluation of the dynamical and statistical properties of multiple streams of physiologic and vital sign data has not been undertaken in large numbers of patients before;
we generate and rely on more informed analysis and annotation of patient events by clinicians;
the concerted effort of critical masses of clinicians with quantitative scientists has not previously been systematized in a patient-centered project, one in which the mathematics are optimized for specific clinical findings—too often, the clinical rationale has been molded around the math;
we have developed a large, dedicated and highly flexible computer storage and analysis system;
we are underway with continuous recording and storage of multivariate waveform and vital sign data with 50 patient years of data.

### Preliminary Studies: Heart Rate Characteristics Monitoring in the Neonatal ICU

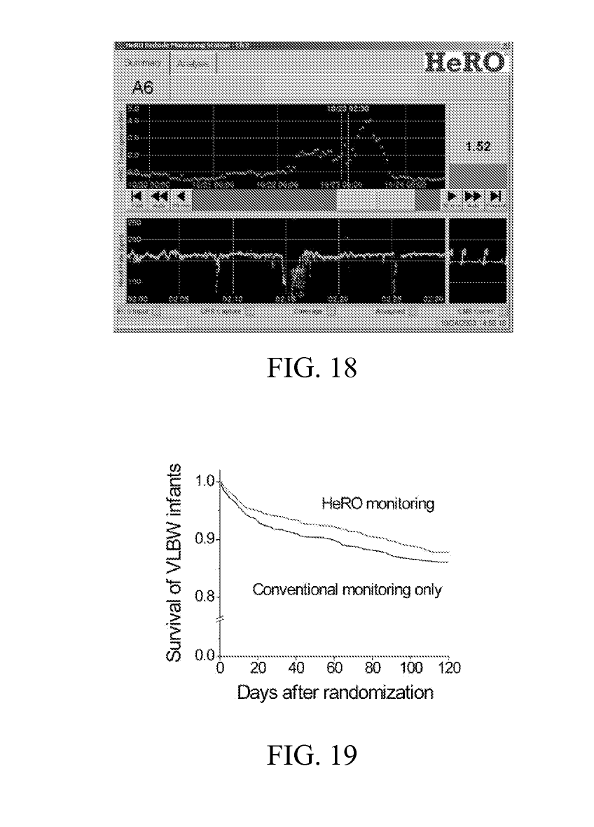

Our initial step was to observe abnormal heart rate characteristics (HRC) in the hours before clinical diagnosis of sepsis (8). The HRC were reduced variability and transient decelerations (FIG. 18) and, while obvious to the eye, they were not detectable using standard HRV measures. We concluded that continuous HRC monitoring might lead to earlier diagnosis of neonatal sepsis.

Much work has followed. We developed new methods for detecting reduced variability and transient decelerations, which we call sample entropy and sample asymmetry (48, 50, 71, 72). We used them, along with standard deviation, to characterize continuous HR records in the UVa and Wake Forest (WFU) NICUs (49, 52). We used multivariable logistic regression to make a predictive model for neonatal sepsis, and found it to be highly significantly associated with impending illness. We call the output of the model the “HRC index”, or the HeRO score (FIG. 18).

Most importantly, we have completed a large RCT on the impact of HeRO monitoring on outcomes. Remarkably, we found reduced mortality in monitored VLBW infants (FIG. 19; HR 0.78, CI 0.64 to 0.99, p<0.05), especially in extremely low birth weight infants (ELBW, <1000 g, HR 0.74, CI 0.57 to 0.95, p<0.02). To save 1 life required monitoring 48 VLBW infants, 23 ELBW infants, or 16 VLBW infants with a sepsis episode (31).

We evaluated the diagnostic accuracy of abnormal HRC index measurements in more than 1000 infants from the UVa and WFU NICUs (73) and in 3000 randomized very low birth weight (<1500 g, VLBW) infants using predictiveness curves (FIG. 21) (74, 75). The data points are the observed fold-increase in risk for each decile of measured HeRO scores, which are plotted as the solid lines. These plots show very good agreement between the expected and observed risks in 2 large populations over the past 10 years, from 9 NICUs. Note the large increase in probability of imminent illness in the highest quintile compared with the lowest—this shows that HeRO scores do well at distinguishing patients at highest risk. The Preliminary Results in STICU patients below are similar.

### Preliminary Studies: Methods to View and Study Data

Since data collection began, there are data on nearly 1400 infants, more than 300 of them VLBW. The group has developed tools for visualizing data at several levels. They array clinical data and extracted monitoring parameters along a time axis for the entire population. In each, the horizontal axis is time in days since birth.

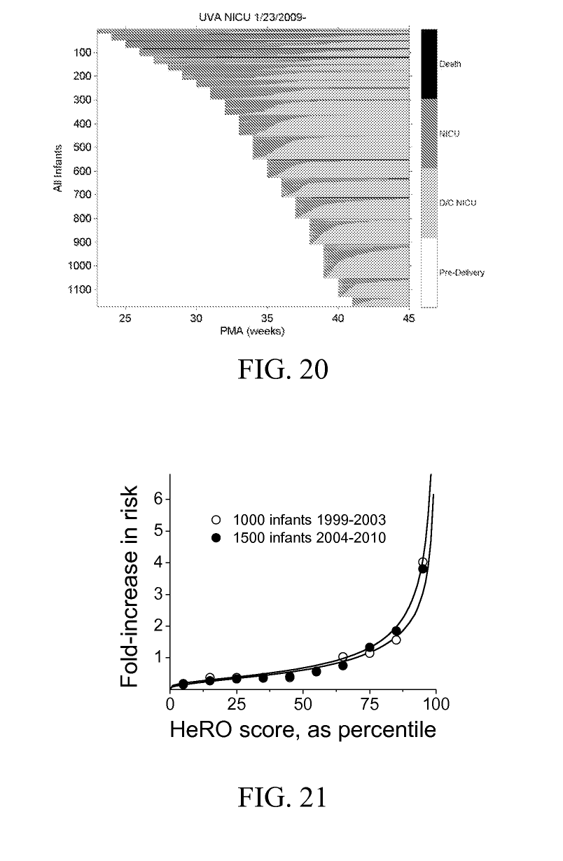

1. Natural history of NICU admissions. The graphic in FIG. 20 represents each infant as a horizontal line extending from birth to discharge in terms of post-menstrual age. Each row is an individual patient with the period of hospitalization marked in dark gray.

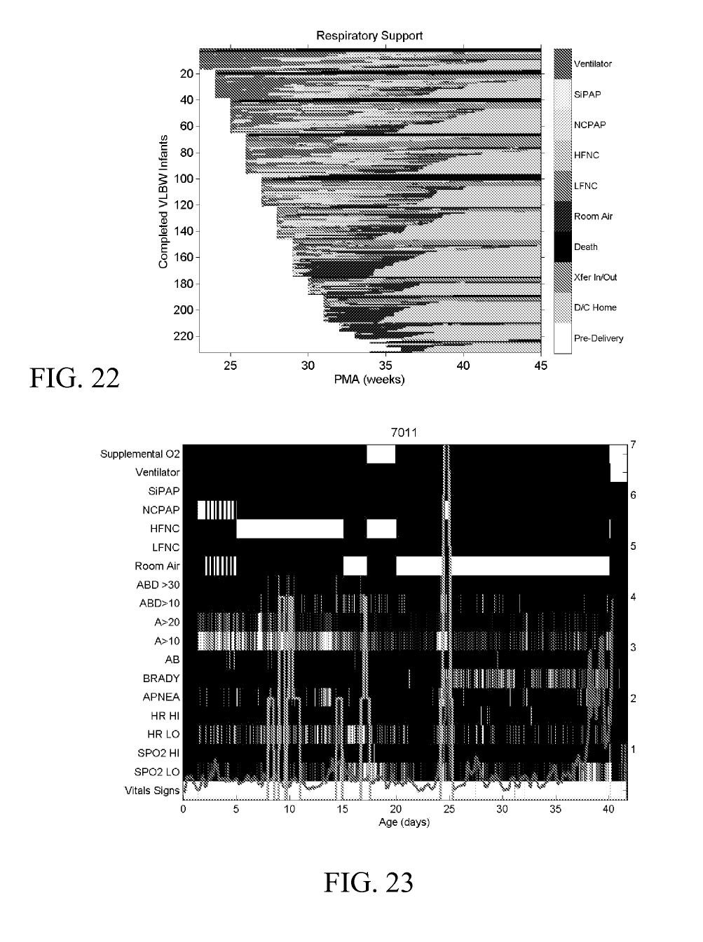

2. Heat map of respiratory support in VLBW infants (FIG. 22) entered by hand into the database, is color-coded. More premature infants receive more intense modalities, and for longer. Note that many infants of GA >31 weeks require no support for much of the NICU stay.

FIG. 23 is an event map of respiratory support in a VLBW infant who died of NEC. Apnea events were detected using our new algorithms described below. The right vertical axis relates to the green line (number of ABD30 events in past 24 hours) and the red line (HeRO score in fold-increase in risk of sepsis in next 24 hours). The horizontal axis is NICU stay in days. The left vertical axis is labeled categorically:

NCPAP, nasal CPAP;
HFNC and LFNC, high- and low-flow nasal cannula;
ABD>30 or >10, central Apneas with Bradycardia and O₂ Desats lasting >30 or >10 seconds;
A>20 and >10, Apneas lasting >20 and >10 seconds;
AB, Apnea and Bradycardia nursing sheet entry; BRADY and APNEA, monitor alarms;
HR HI and LO, monitor alarms for high and low HR; SPO2 HI and LO, monitor alarms for high and low O₂ saturation.

These displays are very useful tools. Clinicians use them to assess individual patients and relate results to clinical events. In joint meetings, clinicians and quantitative scientists use them to assess population trends and to develop summary measures.

### Preliminary Results: Infections in the ICU are Preceded by Abnormal Cardio-Respiratory Control

Our work in adults has begun in earnest in the Surgical/Trauma/Burn ICU, where our colleague R Sawyer has an annotated research database of surgical infections (76, 77). We have collected complete bedside monitor information since May 2010 in the 12-bed unit, and used our grid computing cluster to test the hypothesis that cardiorespiratory control changes within 24 hours of the diagnosis of infection.

A preliminary analysis was performed on 42 ICU patients, 19 of whom had 22 infections diagnosed. The clinical data were obtained prospectively, and complete monitor data comprising more than 6 patient-months were available for analysis. The analysis was carried out in two parts. First, we examined the relationships of known abnormalities of cardiorespiratory control with imminent infection—these included descriptive statistics of the three vital sign measures, heart rate, respiratory rate and O₂ saturation. At every 15 minutes, we calculated mean, S.D., median, 10th and 90th percentiles for the preceding 30 minutes. We also calculated the first difference time series of each, and all of their descriptive statistics. Data from within 24 hours of the diagnosis comprised 2% of the total dataset. We used univariate and multivariate logistic regression methods to test for association.

Each vital sign measure had significant association with the outcome of infection, with p<0.05. The lowest p values were found for respiratory rate variability and several measures from the heart and respiratory rate first difference series. We selected heart rate variability and respiratory rate variability as logical choices for a bivariate model, and found the highly significant results shown in the Table. The ROC area of 0.71 is similar to that of HRC models in University of Virginia NICU.

Second, we tested the crucially important hypothesis that a new measure would add important information. We tested the presence of intermittent strong coupling of heartbeats to breaths using the entropy of beat density histograms above. The results are shown in the Table below, and demonstrate a large increase in the ROC area to 0.76, with a significant p value for the new measure in the 3-variable model. (p values refer to individual coefficients in the model, and are interpreted as showing the contribution of significant independent information.)

| | HR | RR | Cou- | p | p | p | |
|---|---|---|---|---|---|---|---|
| | S.D. | S.D. | pling | HR SD | RR SD | Coupling | ROC |
| Model 1 | x | x | | p < 0.01 | p < 0.01 | | 0.71 |
| Model 2 | x | x | x | p < 0.01 | p < 0.01 | p < 0.03 | 0.76 |

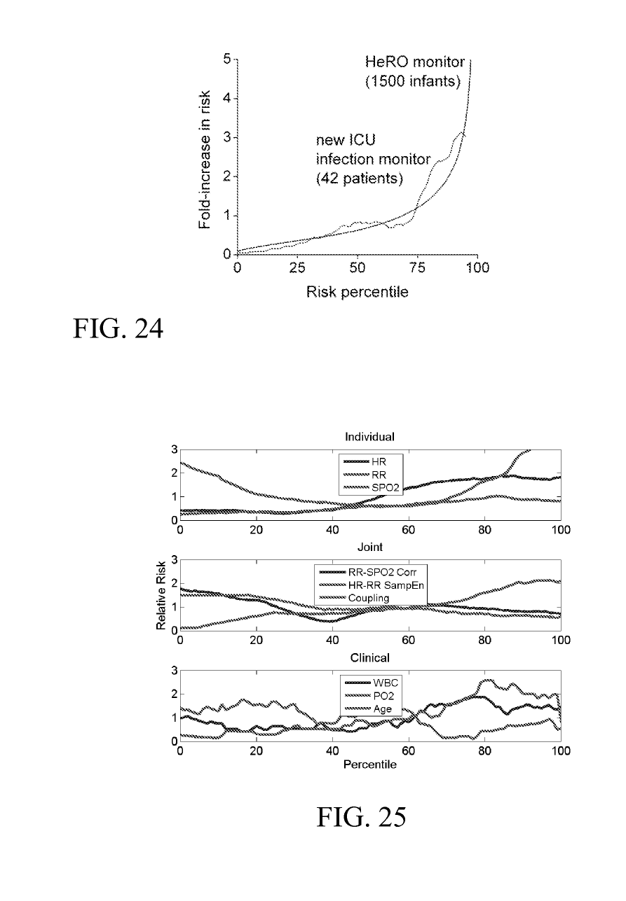

The predictiveness curve for the 3-variable predictive model is shown in red in FIG. 24, along with the predictiveness curve of HRC monitoring in the NICU in blue. There is a similarity despite the very large difference in sample sizes.

We draw 3 conclusions. The first is that infections in adults in the ICU are heralded by changes in cardiorespiratory control—all of the data analyzed were generated prior to clinical signs of illness. The second is that conventional measures can detect these changes, and an early estimate of their accuracy shows it on a par with that of HeRO monitoring in the NICU, a life-saving strategy. The third and most important conclusion is that new measures designed by a team of clinicians and quantitative scientists improve the predictive performance very significantly. This fits with our experience with HeRO monitoring, where the non-linear dynamical measure called sample entropy that we developed is a major contributor to the illness prediction.

### Preliminary Studies: Early Detection of Respiratory Decompensation in Adults Leading to Urgent Unplanned Intubation

Mechanical ventilation can be life-saving, but comes at a cost. The most common complication is ventilator-associated pneumonia (VAP), with incidence up to 28%, increased costs and duration of ventilation and hospital stay and attributable mortality 8 to 10%. Prevention of VAP and other complications of mechanical ventilation begins with avoidance of endotracheal intubation whenever possible, justifying new strategies to detect earlier stages of respiratory decompensation where non-invasive therapies might be effective.

In the Surgical/Trauma ICU, we identified 28 cases of placement of an endotracheal tube and mechanical ventilation because of the onset of respiratory or cardiac failure manifested by severe respiratory distress, hypoxia, hypercarbia, or respiratory acidosis. We collected all monitor data and calculated step-wise:

### Step 1. Individual Measures of Vital Signs.

FIG. 25 shows that increases in HR and RR, and a fall in O₂ saturation are associated with increased risk of urgent intubation. These are expected changes, and the logistic regression model using these changes alone has ROC area 0.76.

### Step 2. Joint Measures.

Both the correlation coefficient (blue) and coupling (red) fall and cross-sample entropy (green) rises with imminent decompensation, all expected with uncoupling of organs in illness. The logistic regression model has ROC area 0.58.

### Step 3. Clinical Measures.

Increasing age and white blood cell count (WBC), and falling pO₂ are associated with increased risk, and the ROC area is 0.68.

### Step 4. Combined Models.

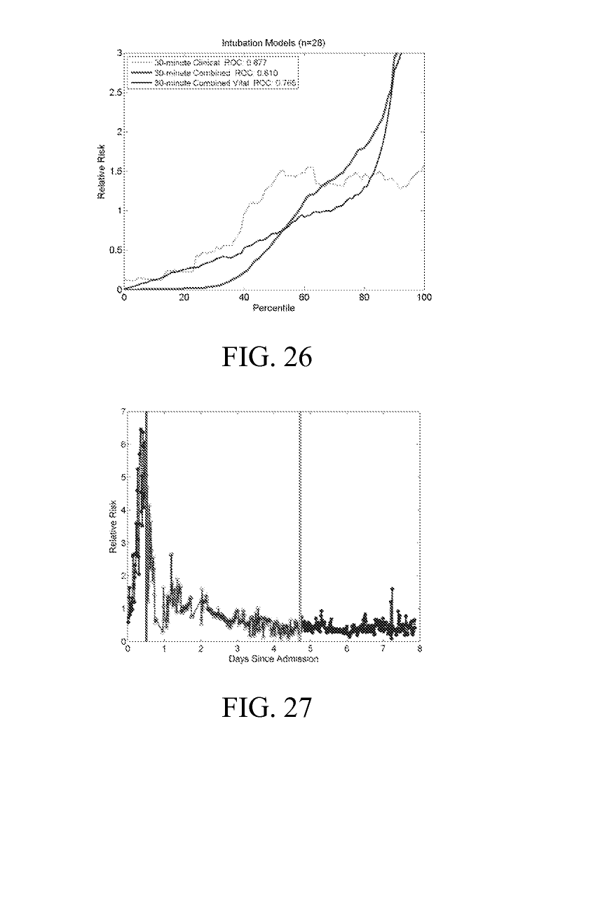

FIG. 26 shows the results of the 1st and 3rd models, and a composite regression model that uses the output of each as separate predictor variables. The overall ROC area is 0.81. Remarkably, patients with model output values in the bottom quartile were not ever intubated in the next 24 hours. This is to some extent an artifact of the small data set, but points to the possibility of clinical utility.

### A Multi-Parameter Statistical Model Predicts Urgent Unplanned Intubation in Medical ICU Patients

Rationale:

ICU patients who develop respiratory decompensation and undergo urgent, unplanned intubations are at risk for complications including cardiac arrest, severe hypotension, and ventilator-associated pneumonia. We hypothesized that multivariable statistical analysis of available ICU bedside monitoring data would allow early detection of these events.

Methods:

We recorded vital signs (heart rate, respiratory rate, and oxygen saturation) every 2 seconds from patients in a 16 bed medical ICU (MICU) and retrospectively identified occurrences of respiratory decompensation resulting in urgent unplanned intubations over a 6-month period. Means and standard deviations of vital signs were calculated every 15 minutes on windows of 30 minute long observations. We excluded periods of mechanical ventilation and patients who had “Do-Not-Intubate” orders. Stepwise logistic regression modeling adjusted for repeated measures was employed to generate multivariable predictive models. The outcome of interest was the 24 hours prior to intubation.

Results:

462 admissions of 418 patients were monitored. 292 monitored patients were at risk for urgent intubation, and we analyzed 452 ventilator-free patient-days in which 28 urgent intubations occurred in 26 patients. Average time monitored before intubation was 1.93 days. Median time monitored before intubation was 0.80 days. Rising heart rate, falling heart rate variability and systolic blood pressure, rising blood pressure and oxygen saturation variability were independently predictive of intubation. A model incorporating these 5 parameters had ROC area of 0.764.

FIG. 27 shows the time course of the model prediction for a 25-year-old man with rapidly increasing pleural effusion due to decompensated hepatic failure. The y-axis is the fold-increase in risk of urgent intubation compared to the MICU average. The vertical red and green lines show times of intubation and extubation, respectively. Prior to intubation, the risk estimate rises. After drainage of the effusion, the risk estimate falls.

Conclusions:

In MICU patients, a statistical model incorporating parameters derived from readily available cardio-respiratory monitoring measurements predicts need for urgent unplanned intubation in the next 24 hours.

Table 1 below provides univariate analyses of vital signs and vital sign variability, and Table 2 below provides Multivariate analysis, with regression coefficient±standard error.

TABLE 1
Univariate analyses of vital signs and vital sign variability

| Variable | P-Value | ROC |
|---|---|---|
| Heart Rate | 0.049528 | 0.640 |
| Respiratory Rate | 0.094920 | 0.599 |
| Pulse Oximetry | 0.000328 | 0.600 |
| Systolic Blood Pressure | 0.002801 | 0.637 |
| Diastolic Blood Pressure | 0.087855 | 0.596 |
| Mean Blood Pressure | 0.015627 | 0.624 |
| Heart Rate Variability | 0.084003 | 0.584 |
| Respiratory rate Variability | 0.743062 | 0.511 |
| Pulse oximetry Variability | 0.000000 | 0.635 |
| Systolic Blood Pressure Variability | 0.000000 | 0.594 |
| Diastolic Blood Pressure Variability | 0.000353 | 0.564 |
| Mean Blood Pressure Variability | 0.000161 | 0.592 |

TABLE 2
Multivariate analysis, with regression coefficient ± standard error

| Variable | Coefficient | Chi-square | P-value |
|---|---|---|---|
| Systolic Blood Pressure | −0.024 ± 0.008 | 8.78 | 0.00305 |
| Heart Rate | 0.019 ± 0.010 | 3.48 | 0.06211 |
| Blood Pressure Variability | 0.079 ± 0.017 | 20.62 | 0.00001 |
| Heart Rate Variability | −0.14 ± 0.061 | 5.62 | 0.01773 |
| Pulse Oximetry Variability. | 0.21 ± 0.03 | 44.45 | 0 |

Generally, our targets should have subclinical prodromes, and early treatment should improve outcomes. While the specific clinical diagnoses and scenarios differ among the ICUs, there are recurring themes. One is sepsis. This is common in all of the units and wards, and especially important because of the added morbidity and mortality risks. Moreover, hospital-acquired sepsis is adjudged to completely avoidable (!) and thus the new costs incurred will not be reimbursed by third-party payors. This has potentially very serious impact on the bottom line of many small to mid-size hospitals. Caught very early, though, sepsis many not be such a problem. The mortality rises with every hour of delay in starting antibiotics, implying that early detection may dramatically limit the severity and burden of the illness.

Another is respiratory decompensation leading to unplanned intubation, especially in the setting of recent extubation. In addition to the personal discomfort of mechanical ventilation, there is the risk of ventilator-associated pneumonia, a diagnosis with high morbidity and mortality. Better detection of early phases of respiratory decompensation may lead to prompt trials of bronchodilators, supplemental oxygen, or more aggressive though still non-invasive ventilatory modalities and thus to avoidance of incubation altogether.

The Table below shows the ICUs involved, the numbers and approximate numbers of admission yearly, and the estimated density of events. Black signifies 50 per year, dark grey signifies 25 per year, and light grey signifies 10 per year based on estimates of the clinicians in the study.

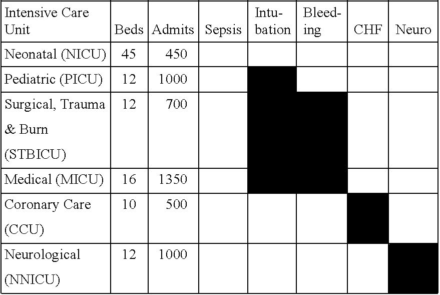

In addition, we will acquire data from monitored wards—often, ICU patients graduate to these beds, and their vulnerability persists.

### Approach: Computer Methods

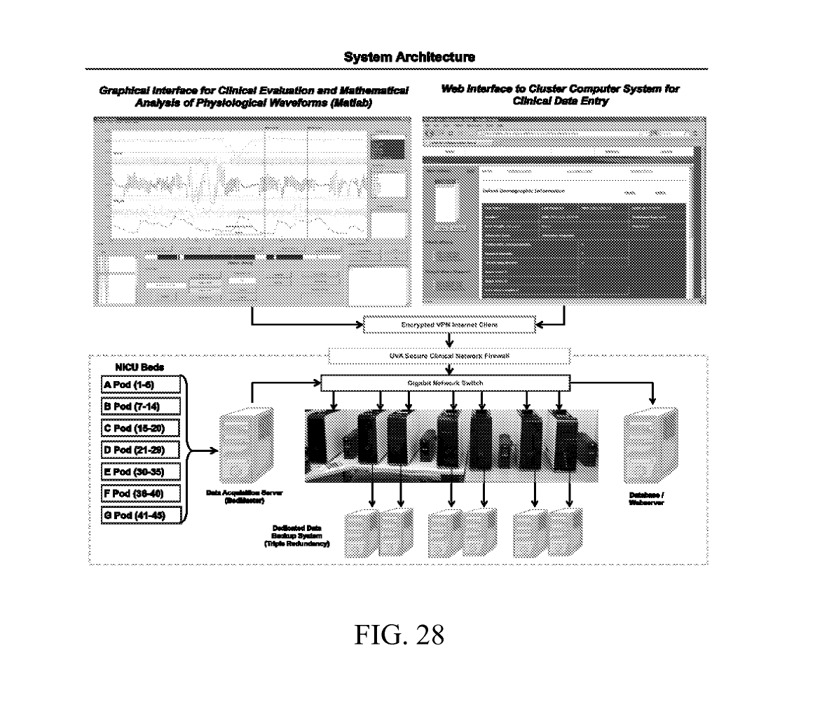

We have been storing digital waveforms from as many as 75 beds, a partial sample of the 200 ICU and 100 monitored ward beds. We store 3 EKG leads, respiratory impedance waveform, and the O₂ saturation signal along with GE monitor-derived bedside alarms and vital signs—about 75 MB per bed per day. Any signal displayed on the bedside monitor is automatically captured—this is relevant to the Neurological ICU where, for example, intracranial pressure waveforms are important. FIG. 28 shows system architecture of dedicated network storage and processing system in which waveforms are archived. The cluster consists of 10 desktop and workstation PCs with a total of 80 processing cores, 40 GB RAM, and 100 TB storage. We use grid-computing and parallel processing. The system was custom-built with help of UVa Information and Technology Center, Health System Computing Services, and the Cardiology computer group. The cluster is hosted inside the UVa secure clinical network behind two firewalls prevent unauthorized access.

Monitor data are downloaded and processed nightly. We use the BedMaster patient monitoring system (Excel Medical, Jupiter, Fla.) to record waveform and vital sign files. These data have no Personal Health Information but are labeled by bed number and a coded timestamp (Patient bed assignments are available from the UVa Clinical Data Repository and reflect the time of the physician orders to admit, discharge or move infants, and are considered very reliable). Data acquisition is interrupted for 4 seconds to initiate the file transfer process. We convert the proprietary data files to a binary format that can be accessed using C/C++ or Matlab. Meta-data, clinical tags, error logs, file logs, and cluster status messages are recorded in a separate MySQL database.

Clinical data such as patient demographic information, ventilator support, and medications, are also entered through a web-based interface. Information entered into the clinical database can be used to automatically apply appropriate clinical tags to sections of the physiological waveforms. Clinical observations can be directly applied to the waveform data with user-defined tags showing event starts and stops. Users jump to the previous or next clinical event with mouse clicks. The graphical interface software allows the user to construct plug-ins of mathematical algorithms to analyze the data currently displayed in the viewing window. Worthy algorithms are then applied to the entire database, and results are available for displays and event tags.

### Approach: Mathematical and Statistical Methods

The data set is large and complicated—waveforms, vital signs, lab tests collected at up to 240/sec!—but the output is to be very simple indeed—an hourly estimate of the fold-increase in risk of imminent bleeding, infection or intubation. We need to extract parameters and to combine them. We extract:

Time-domain parameters, such as the mean and variance to estimate the center and the width of the distributions. Most observations during illness in adults, including those with trauma are of reduced HRV (78, 79) measured is standard ways (80). We will also use our sample asymmetry measure (50), which gives much the same information as the skewness, or third moment (8).

Frequency-domain parameters, or band specific variances. An incontrovertible finding is of reduced sinus arrhythmia during illness, reflected as a reduced area under the spectrum at the respiratory frequency. Moorman and Lake have experience in this area (47).

Phase domain, in which the instantaneous phase of waveforms are found using the Hilbert transform. This is a novel application, and results in phase interaction plots that quantify the heart rate impact of breaths at different points of the cardiac cycle. For example, the coincidence of a heartbeat and the beginning of expiration results in more dramatic slowing.

Signal quality quantifies the noise in the signals, allowing, for example, the computationally intensive phase domain calculations to be reserved for the quietest data.

Apnea detection—the core idea is to remove the cardiac component of the chest impedance signal, which becomes dominant in apnea and can even be counted as breaths.

Entropy estimation using sample entropy and the coefficient of sample entropy, which we have recently developed as a detector of atrial fibrillation in very short—12 beats—heart rate time series. We will test the idea that changes in entropy of the heart rate and other time series is altered as illness develops (16). Moorman and Lake have much experience in this area (48, 71, 72).

Deceleration (or acceleration) detection using a novel wavelet-transform-based algorithm that we developed for neonatal sepsis detection (7). The algorithm is readily adapted to detect the accelerations that we identified in preliminary inspection of trauma ICU data.

We combine them using multivariate statistical methods, such as logistic regression (this is the basis of the HRC index for the NICU)(49, 52, 81), k nearest neighbor analysis (58), neural nets, and other techniques. Generally, 50 events allow for a predictive model with 5 predictive variables and 95% Cl of 0.3 around the ROC area. We designate the 24 hours prior to the event as the outcome of interest. Thus the output of the model is the probability of an event in the next 24 hours, a truly predictive result. We divide by the average probability of the event, and present the clinician with the fold-increase in risk of an upcoming event. Stukenborg and Lake have much experience in statistical modeling and analysis of this kind.

The sample size is estimated based on our experience and the accuracy of ROC areas as measured by the width of the confidence interval. We judge a width of 0.1 or less to be sufficiently accurate. Bootstrapped confidence intervals for ROC area are determined by resampling the population 1000 times with replacement and reporting the 2.5^{th} and 97.5^{th} percentiles. We found that for 149 infants and 110 events of sepsis, the ROC area was 0.75 and the 95% Cl was 0.68 to 0.76, or width of 0.08. From this, we conclude that 100 events or more are necessary for confident estimation of ROC area.

### Multidimensional and BigData Aspects and Approaches

Prediction of imminent and remote patient outcomes from genetic, clinical and physiologic databases represents a new and unsolved challenge for clinicians and their patients. We describe techniques and concepts to successfully exploit the explosion of information that, when taken in proper quantity, perspective and context, allows improved patient outcomes through prediction and early detection.

A key aspect is the incorporation of multiple signals and datasets of differing sampling rates. For example, the genomic sequence is sampled only once, but other -omic datasets are likely to change with circumstance such as aging, infection, cancer, vascular disease, acute or chronic organ failure, or other acute or chronic illness. On faster time scales, sampling of electrophysiological signals from the heart (ECG) or brain (EEG) proceeds at hundreds or thousands of Hz. Other clinically ubiquitous data such as laboratory tests are drawn rarely in ambulatory patients but with great—though not necessarily regular—frequency in the hospital, particularly in the intensive care units.

The general class of solutions is to weight observations by time, scale, and past experience of their relationship to clinical outcomes. Conventional approaches that are readily applicable to modern data sets include, for example, regression—here, the weighting of observations is achieved through estimation of coefficients in a linear combination of observed parameters using datasets from patients with known outcomes.

The invention includes new practices for very large and highly multidimensional data sets consisted of TB and PB size databases populated with genomic, proteomic, metabolomic and other similarly detailed libraries of individual genetic, physiologic and metabolic profiles. Prediction of future events, remote or imminent, can be based on statistical techniques including but not limited to multivariable regression, neural nets, Bayesian nets, other multivariable approaches. A particularly useful approach is k nearest neighbor analysis of in highly dimensional yet very densely populated neighborhoods, or in smaller neighborhoods refined to include only genotypically similar subjects.

In the ICU, ER and hospital setting, this leads to a clinician tool to forecast likely adverse outcomes. As an example, consider seeing a patient knowing the n most likely causes of death in the next m hours, and the individual outcome probabilities. In the ambulatory setting, this leads to a slightly different clinician tool to forecast likely adverse outcomes. As an example, consider seeing a patient knowing the n most likely causes of death in the next m years, and the individual outcome probabilities.

### Neonatal ICU

Aim:

Develop predictive models for central apnea, and enhance the existing predictive model for sepsis

Rationale:

Neonatal apnea occurs in nearly all with birthweights less than 1000 gms (3). Apneas are not predictable, and most neonatologists do not discharge preterm babies to home prior to an apnea-free period of about one week (2). Defining events for these “apnea countdowns” is imprecise and often inaccurate, and, despite continuous electronic monitoring, we still rely on uncalibrated bedside records. False-positive episodes result in unnecessary, expensive delays. Detection failures, on the other hand, may result in release of infants at risk of severe apnea and even sudden infant death syndrome.

The problem of neonatal sepsis is described in detail in the section “Approach: proof of principle” above.

### Pediatric ICU

Aim:

Develop predictive models for respiratory decompensation leading to urgent, unplanned intubation

Rationale:

The Pediatric ICU is the nerve center for advances in diagnosis and treatment of acutely ill children, and those recovering from surgery, particularly cardiac. The population is diverse, and consists of medical, and general and cardiac surgical patients. The post-op patients arrive intubated, and timing of extubation is critically important. Too quickly and there is the risk of respiratory deterioration and the need to re-intubate. Too slowly and there is the risk of ventilator-associated pneumonia and other complications of mechanical ventilation. Each of these unfavorable outcomes should have subclinical phases—of respiratory distress in the too-quickly extubated patient, and of infection in the too-slowly one.

### Surgical, Trauma and Burn ICU

Aim:

Develop predictive models for bleeding, sepsis and unplanned intubation in surgical patients

Rationale:

Bleeding is an important cause of sub-acute potentially catastrophic illness in the trauma population, and accounts for 30-40% of injury-related deaths. Early transfusion might avoid circulatory shock or acute myocardial infarction, and earlier investigation for bleeding sources might lead to intervention, including operative, at times when the patient has not deteriorated and is better able to withstand the procedure.

Infection is arguably the most common and modifiable cause of late death after injury, and there are multiple potential sources. A current concept is the most life-threatening aspects of sepsis are due not to the infecting organisms but rather to an exaggerated immune response, the systemic inflammatory response syndrome. This has been an extremely useful framework for understanding why antibiotics are not curative unless given very early, and underscoring the need for early detection.

Respiratory decompensation leading to urgent, unplanned intubation results in increases in length of stay and mortality. Early detection could lead to interventions such as bronchodilators or antibiotics that might prevent bronchospasm or infection from getting out of control.

### Neurological ICU

Aim:

Develop predictive models for deterioration after brain injury and intracranial hemorrhage

Rationale:

Acute neurologic deterioration is common here, and a new paradigm of brain injury is a systemic inflammatory process with a sub-clinical prodrome. Early detection can lead to more aggressive measures such as placement of an intracranial pressure monitor, osmotic diuresis and even craniotomy. Prior studies have identified systemic inflammation as a key component of the response to TBI. The pro-inflammatory cytokines IL-6 and IL-12 rise after trauma, and non-survivors had much higher levels. As in sepsis, abnormal profiles of circulating cytokines contain information about the severity and ultimate outcomes in TBI, and offer a foundation for strategies for early diagnosis.

A signature of decreasing blood flow and increasing ICP will be sought in the physiological data, especially the universally available heart rate, respiratory rate and O₂ saturation. The ICP records themselves will be inspected for prodromes of acute severe increases. Goldstein and coworkers have shown altered entropy near spikes, justifying the use of the non-linear dynamical methods that we propose. Since increases in ICP are related to blood flow, we will incorporate a regional blood flow measure when possible to the array of vital sign and waveform data, especially in patients with subarachnoid hemorrhage at risk of vasospasm.

### Coronary Care Unit

Aim:

Develop predictive modeling for congestive heart failure (CHF) exacerbation

Rationale:

The Coronary Care Unit of today is unrecognizable from the original concept of a site for specialized care of acute myocardial infarction. Patients today have much more advanced and complex heart disease, and the CCU becomes home for severe, unexpected exacerbation of CHF. Worsening heart failure is difficult to detect in the in- or outpatient setting. Axiomatically, several liters of volume accumulate before clinical signs of edema or symptoms of dyspnea appear.

While we expect in advance that his patients will have reduced HRV, we will look for transient episodes of otherwise unexplained tachycardia, tachypnea and/or hypoxia, or for unexpected patterns of time series of vital signs. In this clinical setting episodes of paroxysmal or sustained arrhythmia such as atrial fibrillation or ventricular tachycardia may hold information about changing status. For this task, Lake and Moorman have recently refined the concept of entropy estimation for atrial fibrillation detection using time series as short as 12 beats. This will be deployed, and VT and AF burdens quantified.

### Medical ICU

Aim:

Develop predictive modeling for sepsis and respiratory decompensation leading to urgent, unplanned intubation

Rationale:

Patients in the MICU often very complex and severe medical illness, and both sepsis and respiratory decompensation commonly tip the scales against full and uncomplicated recovery. There are local and national programs geared toward prevention of nosocomial sepsis, now viewed by CMS as a preventable complication and thus ineligible for reimbursement. If there were not incentive enough, this has led our hospital to aggressively target this diagnosis. Based on our experience in infants, we feel that we can identify the subclinical phases in which treatment may abort the most severe elements of the illness.

Likewise, recent work from Seely in Ottawa shows that variability of the respiratory rate augurs well during spontaneous breathing trials in predicting successful extubation. We will be testing the idea that these metrics will additionally inform early respiratory decompensation.

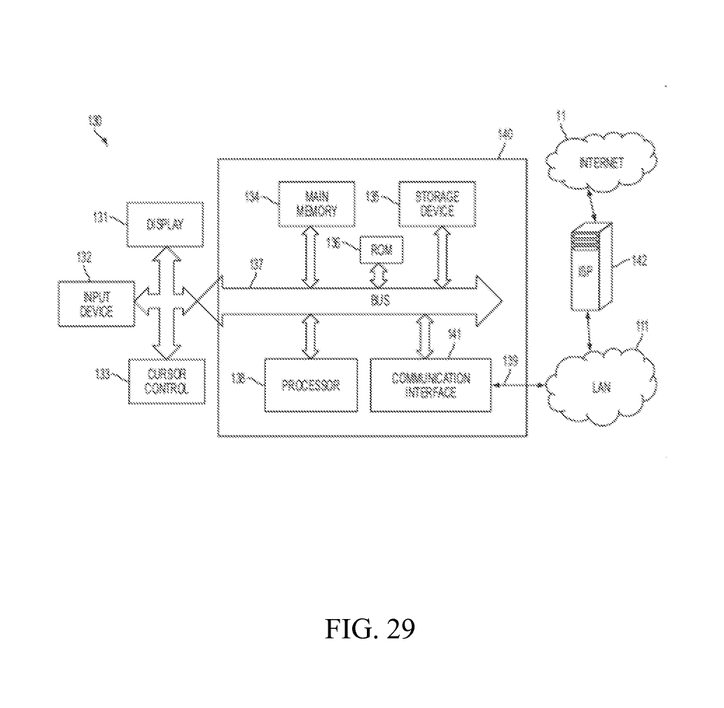

FIG. 29 is a block diagram that illustrates a system 130 including a computer system 140 and the associated Internet 11 connection upon which an embodiment may be implemented. Such configuration is typically used for computers (hosts) connected to the Internet 11 and executing a server or a client (or a combination) software. A source computer such as laptop, an ultimate destination computer and relay servers, for example, as well as any computer or processor described herein, may use the computer system configuration and the Internet connection shown in FIG. 29. The system 140 may be used as a portable electronic device such as a notebook/laptop computer, a media player (e.g., MP3 based or video player), a cellular phone, a Personal Digital Assistant (PDA), cardiological, physiological and/or biological acquisition, diagnostic and/or monitor device, an image processing device (e.g., a digital camera or video recorder), and/or any other handheld computing devices, or a combination of any of these devices (as disclosed herein throughout). Note that while FIG. 29 illustrates various components of a computer system, it is not intended to represent any particular architecture or manner of interconnecting the components; as such details are not germane to the present invention. It will also be appreciated that network computers, handheld computers, cell phones and other data processing systems which have fewer components or perhaps more components may also be used. The computer system of FIG. 29 may, for example, be an Apple Macintosh computer or Power Book, or an IBM compatible PC. Computer system 140 includes a bus 137, an interconnect, or other communication mechanism for communicating information, and a processor 138, commonly in the form of an integrated circuit, coupled with bus 137 for processing information and for executing the computer executable instructions. Computer system 140 also includes a main memory 134, such as a Random Access Memory (RAM) or other dynamic storage device, coupled to bus 137 for storing information and instructions to be executed by processor 138.

Main memory 134 also may be used for storing temporary variables or other intermediate information during execution of instructions to be executed by processor 138. Computer system 140 further includes a Read Only Memory (ROM) 136 (or other non-volatile memory) or other static storage device coupled to bus 137 for storing static information and instructions for processor 138. A storage device 135, such as a magnetic disk or optical disk, a hard disk drive for reading from and writing to a hard disk, a magnetic disk drive for reading from and writing to a magnetic disk, and/or an optical disk drive (such as DVD) for reading from and writing to a removable optical disk, is coupled to bus 137 for storing information and instructions. The hard disk drive, magnetic disk drive, and optical disk drive may be connected to the system bus by a hard disk drive interface, a magnetic disk drive interface, and an optical disk drive interface, respectively. The drives and their associated computer-readable media provide non-volatile storage of computer readable instructions, data structures, program modules and other data for the general purpose computing devices. Typically computer system 140 includes an Operating System (OS) stored in a non-volatile storage for managing the computer resources and provides the applications and programs with an access to the computer resources and interfaces. An operating system commonly processes system data and user input, and responds by allocating and managing tasks and internal system resources, such as controlling and allocating memory, prioritizing system requests, controlling input and output devices, facilitating networking and managing files. Non-limiting examples of operating systems are Microsoft Windows, Mac OS X, and Linux.

The term “processor” is meant to include any integrated circuit or other electronic device (or collection of devices) capable of performing an operation on at least one instruction including, without limitation, Reduced Instruction Set Core (RISC) processors, CISC microprocessors, Microcontroller Units (MCUs), CISC-based Central Processing Units (CPUs), and Digital Signal Processors (DSPs). The hardware of such devices may be integrated onto a single substrate (e.g., silicon “die”), or distributed among two or more substrates. Furthermore, various functional aspects of the processor may be implemented solely as software or firmware associated with the processor.

Computer system 140 may be coupled via bus 137 to a display 131, such as a Cathode Ray Tube (CRT), a Liquid Crystal Display (LCD), a flat screen monitor, a touch screen monitor or similar means for displaying text and graphical data to a user. The display may be connected via a video adapter for supporting the display. The display allows a user to view, enter, and/or edit information that is relevant to the operation of the system. An input device 132, including alphanumeric and other keys, is coupled to bus 137 for communicating information and command selections to processor 138. Another type of user input device is cursor control 133, such as a mouse, a trackball, or cursor direction keys for communicating direction information and command selections to processor 138 and for controlling cursor movement on display 131. This input device typically has two degrees of freedom in two axes, a first axis (e.g., x) and a second axis (e.g., y), that allows the device to specify positions in a plane.

The computer system 140 may be used for implementing the methods and techniques described herein. According to one embodiment, those methods and techniques are performed by computer system 140 in response to processor 138 executing one or more sequences of one or more instructions contained in main memory 134. Such instructions may be read into main memory 134 from another computer-readable medium, such as storage device 135. Execution of the sequences of instructions contained in main memory 134 causes processor 138 to perform the process steps described herein. In alternative embodiments, hard-wired circuitry may be used in place of or in combination with software instructions to implement the arrangement. Thus, embodiments of the invention are not limited to any specific combination of hardware circuitry and software.

The term “computer-readable medium” (or “machine-readable medium”) as used herein is an extensible term that refers to any medium or any memory, that participates in providing instructions to a processor, (such as processor 138) for execution, or any mechanism for storing or transmitting information in a form readable by a machine (e.g., a computer). Such a medium may store computer-executable instructions to be executed by a processing element and/or control logic, and data which is manipulated by a processing element and/or control logic, and may take many forms, including but not limited to, non-volatile medium, volatile medium, and transmission medium. Transmission media includes coaxial cables, copper wire and fiber optics, including the wires that comprise bus 137. Transmission media can also take the form of acoustic or light waves, such as those generated during radio-wave and infrared data communications, or other form of propagated signals (e.g., carrier waves, infrared signals, digital signals, etc.). Common forms of computer-readable media include, for example, a floppy disk, a flexible disk, hard disk, magnetic tape, or any other magnetic medium, a CD-ROM, any other optical medium, punch-cards, paper-tape, any other physical medium with patterns of holes, a RAM, a PROM, and EPROM, a FLASH-EPROM, any other memory chip or cartridge, a carrier wave as described hereinafter, or any other medium from which a computer can read.

Various forms of computer-readable media may be involved in carrying one or more sequences of one or more instructions to processor 138 for execution. For example, the instructions may initially be carried on a magnetic disk of a remote computer. The remote computer can load the instructions into its dynamic memory and send the instructions over a telephone line using a modem. A modem local to computer system 140 can receive the data on the telephone line and use an infra-red transmitter to convert the data to an infra-red signal. An infra-red detector can receive the data carried in the infra-red signal and appropriate circuitry can place the data on bus 137. Bus 137 carries the data to main memory 134, from which processor 138 retrieves and executes the instructions. The instructions received by main memory 134 may optionally be stored on storage device 135 either before or after execution by processor 138.

Computer system 140 also includes a communication interface 141 coupled to bus 137. Communication interface 141 provides a two-way data communication coupling to a network link 139 that is connected to a local network 111. For example, communication interface 141 may be an Integrated Services Digital Network (ISDN) card or a modem to provide a data communication connection to a corresponding type of telephone line. As another non-limiting example, communication interface 141 may be a local area network (LAN) card to provide a data communication connection to a compatible LAN. For example, Ethernet based connection based on IEEE802.3 standard may be used such as 10/100BaseT, 1000BaseT (gigabit Ethernet), 10 gigabit Ethernet (10 GE or 10 GbE or 10 GigE per IEEE Std 802.3ae-2002 as standard), 40 Gigabit Ethernet (40 GbE), or 100 Gigabit Ethernet (100 GbE as per Ethernet standard IEEE P802.3ba), as described in Cisco Systems, Inc. Publication number 1-587005-001-3 (6/99), “Internetworking Technologies Handbook”, Chapter 7: “Ethernet Technologies”, pages 7-1 to 7-38, which is incorporated in its entirety for all purposes as if fully set forth herein. In such a case, the communication interface 141 typically include a LAN transceiver or a modem, such as Standard Microsystems Corporation (SMSC) LAN91C111 10/100 Ethernet transceiver described in the Standard Microsystems Corporation (SMSC) data-sheet “LAN91C111 10/100 Non-PCI Ethernet Single Chip MAC+PHY” Data-Sheet, Rev. 15 (Feb. 20, 2004), which is incorporated in its entirety for all purposes as if fully set forth herein.

Wireless links may also be implemented. In any such implementation, communication interface 141 sends and receives electrical, electromagnetic or optical signals that carry digital data streams representing various types of information.

Network link 139 typically provides data communication through one or more networks to other data devices. For example, network link 139 may provide a connection through local network 111 to a host computer or to data equipment operated by an Internet Service Provider (ISP) 142. ISP 142 in turn provides data communication services through the world wide packet data communication network Internet 11. Local network 111 and Internet 11 both use electrical, electromagnetic or optical signals that carry digital data streams. The signals through the various networks and the signals on the network link 139 and through the communication interface 141, which carry the digital data to and from computer system 140, are exemplary forms of carrier waves transporting the information.

A received code may be executed by processor 138 as it is received, and/or stored in storage device 135, or other non-volatile storage for later execution. In this manner, computer system 140 may obtain application code in the form of a carrier wave.

The concept of early detection of sub-acute potentially catastrophic illnesses, and more specifically to detecting abnormal entrainment of waveform and vital sign time series representations of physiological processes may be implemented and utilized with the related processors, networks, computer systems, internet, and components and functions according to the schemes disclosed herein.

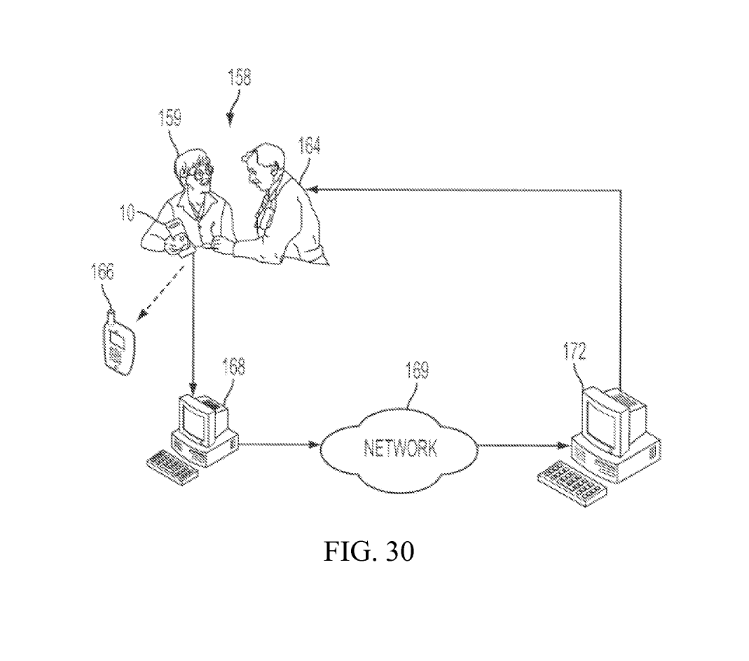

FIG. 30 illustrates a system in which one or more embodiments of the invention can be implemented using a network, or portions of a network or computers.

FIG. 30 diagrammatically illustrates an exemplary system in which examples of the invention can be implemented. Referring to FIG. 30, a clinic setup 158 or the like provides a place for doctors (e.g. 164) or clinician/assistant to diagnose, monitor or treat the patients (e.g. 159) with a cardiological, physiological and/or biological acquisition, diagnostic and/or monitor device(s) 10. Item no. 10 is intended to be a variety of devices or tools and should not be limited by the extent of the specific illustration. The system or component may be affixed to the patient or in communication with the patient as desired or required. For example the system or combination of components thereof—including the cardiological, physiological and/or biological acquisition, diagnostic and/or monitor device, 10, a controller or any other device or component—may be in contact or affixed to the patient through tape or tubing or may be in communication through wired or wireless connections. Such monitor, diagnosis and/or test can be short term (e.g. clinical visit) or long term (e.g. clinical stay, ICU). The device outputs can be used by the doctor (clinician or assistant) for appropriate actions, early detection of sub-acute potentially catastrophic illnesses, and more specifically to detecting abnormal entrainment of waveform and vital sign time series representations of physiological processes, or other appropriate actions. Alternatively, the device output can be delivered to (or data exchanged with) computer terminal 168 for instant or future analyses. The delivery can be through cable or wireless or any other suitable medium. The device output from the patient can also be delivered to a portable device, such as PDA 166. The device outputs can be delivered to (or data exchanged with) a center 172 for processing and/or analyzing. Such delivery can be accomplished in many ways, such as network connection 170, which can be wired or wireless.

In addition to the device outputs, errors, parameters for accuracy improvements, and any accuracy related information can be delivered, such as to computer 168, and/or the center 172 for performing other desired, need or required analyses, diagnosis, or monitoring. This can provide a centralized analyses, database/storage, monitoring or other techniques or components as desired or required.

The following patents, applications and publications as listed below and throughout this document are hereby incorporated by reference in their entirety herein, and which are not admitted to be prior art with respect to the present invention by inclusion in this section.

The devices, systems, compositions, computer readable medium, and methods of various embodiments of the invention disclosed herein may utilize aspects disclosed in the following references, applications, publications and patents and which are hereby incorporated by reference herein in their entirety (and which are not admitted to be prior art with respect to the present invention by inclusion in this section):
1. Goldberger A, Jalife J. Fractal electrodynamics of the heartbeat. In: Mathematical Approaches to Cardiac Arrhythmias. New York: The New York Academy of Sciences; 1990. p. 402-9.
2. Goldberger A L. Is the normal heartbeat chaotic or homeostatic? News. Physiol Sci. 1991; 6:87-91.:87-91.
3. Buchman T G. Physiologic stability and physiologic state. J Trauma 1996; 41:599-605.
4. Buchman T G. Nonlinear dynamics, complex systems, and the pathobiology of critical illness. Curr. Opin. Crit Care. 2004; 10:378-82.
5. Brown T E, Beightol L A, Koh J, Eckberg D L. Important influence of respiration on human R-R interval power spectra is largely ignored. Journal of Applied Physiology 1993; 75:2310-7.
6. Tracey K J. Reflex control of immunity. Nat Rev Immunol 2009; 9:418-28.
7. Flower A A, Moorman J R, Lake D E, Delos J B. Dynamical theory of periodic heart rate decelerations in premature infants. Experimental Medicine and Biology 2010; 235:531-8.
8. Griffin M P, Moorman J R. Toward the early diagnosis of neonatal sepsis and sepsis-like illness using novel heart rate analysis. Pediatrics 2001; 107:97-104.
9. Kattwinkel J, Fanaroff A A, Klaus M H. Bradycardia in preterm infants: indications and hazards of atropine therapy. Pediatrics 1976; 58:494-9.
10. Fairchild K D, Srinivasan V, Moorman J R, Gaykema R, Goehler L. Pathogen-induced heart rate changes associated with cholinergic nervous system activation. American Journal of Physiology 2011; 330:R330-9.
11. Huston J M, Tracey K J. The pulse of inflammation: heart rate variability, the cholinergic anti-inflammatory pathway and implications for therapy. J Intern Med 2011; 269:45-53.
12. Ahmad S, Tejuja A, Newman K D, Zarychanski R, Seely A J. Clinical review: a review and analysis of heart rate variability and the diagnosis and prognosis of infection. Crit Care 2009; 13:232.
13. Ahmad S, Ramsay T, Huebsch L, Flanagan S, McDiarmid S, Batkin I, et al. Continuous multi-parameter heart rate variability analysis heralds onset of sepsis in adults. PLoS One 2009; 4:e6642.
14. Eckberg D L. Human sinus arrhythmia as an index of vagal cardiac outflow. J Appl. Physiol 1983; 54:961-6.
15. Godin P J, Buchman T G. Uncoupling of biological oscillators: a complementary hypothesis concerning the pathogenesis of multiple organ dysfunction syndrome. Crit Care Med. 1996; 24:1107-16.
16. Godin P J, Fleisher L A, Eidsath A, Vandivier R W, Preas H L, Banks S M, et al. Experimental human endotoxemia increases cardiac regularity: results from a prospective, randomized, crossover trial. Critical Care Medicine 1996; 24:1117-24.
17. Lin P W, Nasr T R, Stoll B J. Necrotizing enterocolitis: recent scientific advances in pathophysiology and prevention. Semin Perinatol 2008; 32:70-82.
18. Lin P W, Stoll B J. Necrotising enterocolitis. Lancet 2006; 368:1271-83.
19. Stoll B J. Epidemiology of necrotizing enterocolitis. Clinics in Perinatology 1994; 21:205-18.
20. Hunter C J, Podd B, Ford H R, Camerini V. Evidence vs experience in neonatal practices in necrotizing enterocolitis. J Perinatol 2008; 28 Suppl 1:S9-S13.
21. Lodha A, Asztalos E, Moore A M. Cytokine levels in neonatal necrotizing enterocolitis and long-term growth and neurodevelopment. Acta Paediatr 2009; 99:338-43.
22. Krous H F, Beckwith J B, Byard R W, Rognum T O, Bajanowski T, Corey T, et al. Sudden infant death syndrome and unclassified sudden infant deaths: a definitional and diagnostic approach. Pediatrics 2004; 114:234-8.
23. Malloy M H. SIDS—a syndrome in search of a cause. N Engl J Med 2004; 351:957-9.
24. Guevara M R, Shrier A, Glass L. Phase-locked rhythms in periodically stimulated heart cell aggregates. American Journal of Physiology 1988; 254:H1-H10.
25. Darnall R A, Ariagno R L, Kinney H C. The late preterm infant and the control of breathing, sleep, and brainstem development: A review. Clinics in Perinatology 2006; 33:883-914.
26. Kinney H C, Richerson G B, Dymecki S M, Darnall R A, Nattie E E. The brainstem and serotonin in the sudden infant death syndrome. Annu Rev Pathol 2009; 4:517-50.
27. Shannon R P, Patel B, Cummins D, Shannon A H, Ganguli G, Lu Y. Economics of central line-associated bloodstream infections. Am J Med Qual 2006; 21:7S-16S.
28. Martin G S, Mannino D M, Eaton S, Moss M. The epidemiology of sepsis in the United States from 1979 through 2000. N Engl J Med 2003; 348:1546-54.
29. Rhodes A, Cusack R J, Newman P J, Grounds R M, Bennett E D. A randomised, controlled trial of the pulmonary artery catheter in critically ill patients. Intensive Care Med 2002; 28:256-64.
30. Connors A F, Jr., Speroff T, Dawson N V, Thomas C, Harrell F E, Jr., Wagner D, et al. The effectiveness of right heart catheterization in the initial care of critically ill patients. SUPPORT Investigators. JAMA 1996; 276:889-97.
31. Moorman J R, Carlo W A, Kattwinkel J, Schelonka R L, Porcelli P J, Naverrete C T, et al. Mortality reduction by heart rate characteristics monitoring in very low birthweight neonates: a randomized trial. Journal of Pediatrics 2011; 159:900-6.
32. Martin J A, Hamilton B E, Sutton P D, Ventura S J, Menacker F, Kirmeyer S, et al. Births: final data for 2006. Natl. Vital Stat. Rep. 2009; 57:1-103.
33. Eichenwald E C, Stark A R. Management and outcomes of very low birth weight. N Engl J Med 2008; 358:1700-11.
34. Stoll B J, Hansen N I, Adams-Chapman I, Fanaroff A A, Hintz S R, Vohr B, et al. Neurodevelopmental and growth impairment among extremely low-birth-weight infants with neonatal infection. JAMA. 2004; 292:2357-65.
35. Stoll B J, Hansen N, Fanaroff A A, Wright L L, Carlo W A, Ehrenkranz R A, et al. Late-onset sepsis in very low birth weight neonates: the experience of the NICHD Neonatal Research Network. Pediatrics 2002; 110:285-91.
36. Bone R C. Sepsis and SIRS. Nephrology, Dialysis, Transplantation 1994; 9 Suppl 4:99-103.
37. Finer N N, Higgins R, Kattwinkel J, Martin R J. Summary proceedings from the apnea-of-prematurity group. Pediatrics. 2006; 117:S47-S51.
38. Muttitt S C, Finer N N, Tierney A J, Rossmann J. Neonatal apnea: diagnosis by nurse versus computer. Pediatrics 1988; 82:713-20.
39. Merritt T A, Pillers D, Prows S L. Early NICU discharge of very low birth weight infants: a critical review and analysis. Semin Neonatol 2003; 8:95-115.
40. Darnall R A, Kattwinkel J, Nattie C, Robinson M. Margin of safety for discharge after apnea in preterm infants. Pediatrics 1997; 100:795-801.
41. Ramanathan R, Corwin M J, Hunt C E, Lister G, Tinsley L R, Baird T, et al. Cardiorespiratory events recorded on home monitors: Comparison of healthy infants with those at increased risk for SIDS. JAMA 2001; 285:2199-207.
42. Eichenwald E C, Aina A, Stark A R. Apnea frequently persists beyond term gestation in infants delivered at 24 to 28 weeks. Pediatrics 1997; 100:354-9.
43. Hageman J R, Holmes D, Suchy S, Hunt C E. Respiratory pattern at hospital discharge in asymptomatic preterm infants. Pediatr Pulmonol 1988; 4:78-83.
44. Griffin M P, Scollan D F, Moorman J R. The dynamic range of neonatal heart rate variability. Journal of Cardiovascular Electrophysiology 1994; 5:112-24.
45. Aghili A A, Rizwan u, Griffin M P, Moorman J R. Scaling and ordering of neonatal heart rate variability. Phys Rev Lett 1995; 74:1254-7.
46. Nelson J C, Rizwan u, Griffin M P, Moorman J R. Probing the order within neonatal heart rate variability. Pediatr Res 1998; 43:823-31.
47. Chang K L, Monahan K J, Griffin M P, Lake D, Moorman J R. Comparison and clinical application of frequency domain methods in analysis of neonatal heart rate time series. Ann Biomed Eng 2001; 29:764-74.
48. Lake D E, Richman J S, Griffin M P, Moorman J R. Sample entropy analysis of neonatal heart rate variability. American Journal of Physiology 2002; 283:R789-R97.
49. Griffin M P, O'Shea T M, Bissonette E A, Harrell F E, Jr., Lake D E, Moorman J R. Abnormal heart rate characteristics preceding neonatal sepsis and sepsis-like illness. Pediatr Res 2003; 53:920-6.
50. Kovatchev B P, Farhy L S, Cao H, Griffin M P, Lake D E, Moorman J R. Sample asymmetry analysis of heart rate characteristics with application to neonatal sepsis and systemic inflammatory response syndrome. Pediatr Res 2003; 54:892-8.
51. Cao H, Lake D E, Griffin M P, Moorman J R. Increased nonstationarity of neonatal heart rate before the clinical diagnosis of sepsis. Ann Biomed Eng 2004; 32:233-44.
52. Griffin M P, O'Shea T M, Bissonette E A, Harrell F E, Jr., Lake D E, Moorman J R. Abnormal heart rate characteristics are associated with neonatal mortality. Pediatr Res 2004; 55:782-8.
53. Griffin M P, Lake D E, Bissonette E A, Harrell F E, Jr., O'Shea T M, Moorman J R. Heart rate characteristics: novel physiomarkers to predict neonatal infection and death. Pediatrics 2005; 116:1070-4.
54. Griffin M P, Lake D E, Moorman J R. Heart rate characteristics and laboratory tests in neonatal sepsis. Pediatrics 2005; 115:937-41.
55. Moorman J R, Lake D E, Griffin M P. Heart rate characteristics monitoring in neonatal sepsis. IEEE Transactions in Biomedical Engineering 2006; 53:126-32.
56. Griffin M P, Lake D, Moorman J R. Heart rate characteristics and clinical signs in neonatal sepsis. Pediatric Research 2007; 61:222-7.
57. Addison K, Griffin M P, Moorman J R, Lake D E, O'Shea T M. Heart rate characteristics and neurodevelopmental outcome in very low birth weight infants. J Perinatol. 2009.
58. Xiao Y, Griffin P, Lake D E, Moorman J R. Nearest-neighbor and logistic regression analyses of clinical and heart rate characteristics in the early diagnosis of neonatal sepsis. Med Decis Making 2009; 30:258-66.
59. Fairchild K D, Saucerman J J, Raynor L L, Sivak J A, Xiao Y, Lake D E, et al. Endotoxin depresses heart rate variability in mice: cytokine and steroid effects. American Journal of Physiology 2009; 297:R1019-27.
60. Arias E, MacDorman M F, Strobino D M, Guyer B. Annual summary of vital statistics—2002. Pediatrics 2003; 112:1215-30.
61. Meadow W, Frain L, Ren Y, Lee G, Soneji S, Lantos J. Serial assessment of mortality in the neonatal intensive care unit by algorithm and intuition: certainty, uncertainty, and informed consent. Pediatrics 2002; 109:878-86.
62. Tuzcu V, Nas S, Ulusar U, Ugur A, Kaiser J R. Altered heart rhythm dynamics in very low birth weight infants with impending intraventricular hemorrhage. Pediatrics 2009; 123:810-5.
63. Addison K, Griffin M P, Moorman J R, Lake D E, O'Shea T M. Heart rate characteristics and neurodevelopmental outcome in very low birth weight infants. J Perinatol 2009; 29:750-6.
64. Whitelaw A, Jary S, Kmita G, Wroblewska J, Musialik-Swietlinska E, Mandera M, et al. Randomized trial of drainage, irrigation and fibrinolytic therapy for premature infants with posthemorrhagic ventricular dilatation: developmental outcome at 2 years. Pediatrics 2010; 125:e852-8.
65. Whellan D J, Ousdigian K T, Al-Khatib S M, Pu W, Sarkar S, Porter C B, et al. Combined heart failure device diagnostics identify patients at higher risk of subsequent heart failure hospitalizations: results from PARTNERS HF (Program to Access and Review Trending Information and Evaluate Correlation to Symptoms in Patients With Heart Failure) study. J Am Coll Cardiol 2010; 55:1803-10.
66. Conraads V M, Tavazzi L, Santini M, Oliva F, Gerritse B, Yu C M, et al. Sensitivity and positive predictive value of implantable intrathoracic impedance monitoring as a predictor of heart failure hospitalizations: the SENSE-HF trial. Eur Heart J 2011; 2011:28.
67. Ritzema J, Melton I C, Richards A M, Crozier I G, Frampton C, Doughty R N, et al. Direct left atrial pressure monitoring in ambulatory heart failure patients: initial experience with a new permanent implantable device. Circulation 2007; 116:2952-9.
68. Hunt S A, Abraham W T, Chin M H, Feldman A M, Francis G S, Ganiats T G, et al. ACC/AHA 2005 Guideline Update for the Diagnosis and Management of Chronic Heart Failure in the Adult: a report of the American College of Cardiology/American Heart Association Task Force on Practice Guidelines (Writing Committee to Update the 2001 Guidelines for the Evaluation and Management of Heart Failure): developed in collaboration with the American College of Chest Physicians and the International Society for Heart and Lung Transplantation: endorsed by the Heart Rhythm Society. Circulation 2005; 112:e154-235.
69. Hamid T, Guo S Z, Kingery J R, Xiang X, Dawn B, Prabhu S D. Cardiomyocyte NF-kappaB p65 promotes adverse remodelling, apoptosis, and endoplasmic reticulum stress in heart failure. Cardiovasc Res 2011; 89:129-38.
70. Gordon J W, Shaw J A, Kirshenbaum L A. Multiple facets of NF-kappaB in the Heart: To be or not to NF-kappaB. Circ Res 2011; 108:1122-32.
71. Richman J S, Lake D E, Moorman J R. Sample entropy. Methods Enzymol. 2004; 384:172-84.
72. Richman J S, Moorman J R. Physiological time-series analysis using approximate entropy and sample entropy. Am J Physiol Heart Circ Physiol 2000; 278:H2039-49.
73. Griffin M P, Lake D E, Bissonette E A, Harrell F E, Jr., O'Shea T M, Moorman J R. Heart rate characteristics: novel physiomarkers to predict neonatal infection and death. Pediatrics 2005; 116:1070-4.
74. Gu W, Pepe M S, Huang Y, Janes H, Alonzo T A, Zheng Y, et al. Estimating the diagnostic likelihood ratio of a continuous marker. Biostatistics 2007; 12:87-101.
75. Pepe M S, Feng Z, Huang Y, Longton G, Prentice R, Thompson I M, et al. Integrating the predictiveness of a marker with its performance as a classifier. Am J Epidemiol 2008; 167:362-8.
76. Sawyer R G, Leon C A. Common complications in the surgical intensive care unit. Grit Care Med 2010; 38:S483-93.
77. Hranjec T, Swenson B R, Dossett L A, Metzger R, Flohr T R, Popovsky K A, et al. Diagnosis-dependent relationships between cytokine levels and survival in patients admitted for surgical critical care. J Am Coll Surg; 210:833-44, 45-6.
78. Buchman T G, Stein P K, Goldstein B. Heart rate variability in critical illness and critical care. Curr. Opin. Crit Care 2002; 8:311-5.
79. Morris J A, Jr., Norris P R, Ozdas A, Waitman L R, Harrell F E, Jr., Williams A E, et al. Reduced heart rate variability: an indicator of cardiac uncoupling and diminished physiologic reserve in 1,425 trauma patients. J Trauma 2006; 60:1165-73; discussion 73-4.
80. Electrophysiology TFotESoCatNASoPa. Heart rate variability: standards of measurement, physiological interpretation and clinical use. Circulation 1996; 93:1043-65.
81. Harrell F E, Jr. Regression modeling strategies: with applications to linear models, logistic regression and survival analysis. Berlin: Springer; 2001.
a. U.S. patent application Ser. No. 13/695,243 entitled “SYSTEM, METHOD AND COMPUTER PROGRAM PRODUCT FOR THE ORGANISM-SPECIFIC DIAGNOSIS OF SEPTICEMIA IN INFANTS,” filed Oct. 29, 2012)
b. International Patent Application No. PCT/US2011/034487 entitled “SYSTEM, METHOD AND COMPUTER PROGRAM PRODUCT FOR THE ORGANISM-SPECIFIC DIAGNOSIS OF SEPTICEMIA IN INFANTS,” filed Apr. 29, 2011.)
c. U.S. patent application Ser. No. 12/866,056 entitled “System, Method and Computer Program Product for Detection of Changes in Health Status and Risk of Imminent Illness,” filed Aug. 4, 2010; U.S. Patent Application Publication No. 2010/0324436, Dec. 23, 2010.
d. International Patent Application No. PCT/US2009/033082 entitled “System, Method and Computer Program Product for Detection of Changes in Health Status and Risk of Imminent Illness,” filed Feb. 4, 2009.
e. U.S. patent application Ser. No. 12/724,162 entitled “Method, System and Computer Program Method for Detection of Pathological Fluctuations of Physiological Signals to Diagnose Human Illness,” filed Mar. 15, 2010; U.S. Patent Application Publication No. 2010/0234748, Sep. 16, 2010.
f. U.S. patent application Ser. No. 12/594,842 entitled “Method, System and Computer Program Product for Non-Invasive Classification of Cardiac Rhythm,” filed Oct. 6, 2009; U.S. Patent Application Publication No. 2010/0056940, Mar. 4, 2010.
g. International Patent Application No. PCT/US2008/060021 entitled “Method, System and Computer Program Product for Non-Invasive Classification of Cardiac Rhythm,” filed Apr. 11, 2008.
h. U.S. patent application Ser. No. 10/545,257 entitled “Quantitative Fetal Heart Rate and Cardiotocographic Monitoring System and Related Method Thereof,” filed Aug. 10, 2005; U.S. Pat. No. 7,519,417, issued Apr. 14, 2009.)
i. International Patent Application No. US2004/004113 entitled “Quantitative Fetal Heart Rate and Cardiotocographic Monitoring System and Related Method Thereof,” filed Feb. 12, 2004.
j. U.S. patent application Ser. No. 11/000,630 entitled “Method and apparatus for the early diagnosis of sub-acute, potentially catastrophic illness,” filed Dec. 1, 2004; U.S. Pat. No. 7,774,050, issued Aug. 10, 2010.
k. U.S. patent application Ser. No. 09/770,653 entitled “Method for the early diagnosis of sub-acute, potentially catastrophic illness,” filed Jan. 29, 2001; U.S. Pat. No. 6,856,831, issued Feb. 15, 2005.
l. U.S. patent application Ser. No. 09/668,309 entitled “Method and apparatus for the early diagnosis of sub-acute, potentially catastrophic illness,” filed Sep. 25, 2000; U.S. Pat. No. 6,330,469, issued Dec. 11, 2001.
m. International Patent Application No. US99/05687 entitled “METHOD AND APPARATUS FOR THE EARLY DIAGNOSIS OF SUB-ACUTE, POTENTIALLY CATASTROPHIC ILLNESS,” filed Mar. 17, 1999.
n. U.S. patent application Ser. No. 09/271,279 entitled “Method and apparatus for the early diagnosis of sub-acute, potentially catastrophic illness,” filed Mar. 17, 1999; U.S. Pat. No. 6,216,032, issued Apr. 10, 2001.
o. U.S. patent application Ser. No. 10/069,674 entitled “Method and Apparatus for Predicting the Risk of Hypoglycemia,” filed Feb. 22, 2002; U.S. Pat. No. 6,923,763, issued Aug. 2, 2005.
p. International Patent Application No. US00/22886 entitled “METHOD AND APPARATUS FOR PREDICTING THE RISK OF HYPOGLYCEMIA,” filed Aug. 21, 2000.
q. International Patent Application No. PCT/US2002/005676 entitled “Method and Apparatus for the Early Diagnosis of Sub-acute, Potentially Catastrophic Illness,” filed Feb. 27, 2002.)
r. U.S. patent application Ser. No. 09/793,653 entitled “Method and Apparatus for the Early Diagnosis of Sub-acute, Potentially Catastrophic Illness,” filed Feb. 27, 2001; U.S. Pat. No. 6,804,551, issued Oct. 12, 2004.

What is claimed is:

1. A computer-implemented method for advance detection of sub-acute, potentially catastrophic illness in a patient from abnormal entrainment of multidimensional time series representations of physiological processes of the patient, comprising:
calculating mathematical characteristics of relationships between a plurality of simultaneous time series representations of physiological processes of said patient in a plurality of domains;
determining cross-measures of said time series representations in specified ranges of time lags and frequency bands as functions of at least two simultaneous time series representations of physiological processes;
calculating rates of change of said cross-measures over a specified time window of predefined length;
identifying a rank order of said cross-measures with respect to each other;
identifying a rank order of said cross-measures with respect to their expected distributions;
determining the concordance or discordance among said time series representations;
determining the percentile or rank of the determined cross-measures with respect to a database of cross-measures;
calculating a probability of an illness from a predefined multivariable statistical model that employs at least some observed cross-measure parameters and/or their percentile or rank; and
displaying said probability of illness on a display device.

2. The computer-implemented method of claim 1, wherein said plurality of time domains in said calculating mathematical characteristics includes at least two of: time domain, frequency domain, wavelet domain, non-linear domain, phase domain, and information domain.

3. The computer-implemented method of claim 2, wherein mathematical characteristics of the time domain include at least one of autocorrelation, cross-correlation and covariance.

4. The computer-implemented method of claim 2, wherein mathematical characteristics of the frequency domain include at least one of frequency spectra using a Fourier transform, Lomb periodogram, cross-spectra, coherence, or transfer functions.

5. The computer-implemented method of claim 2, wherein mathematical characteristics of the wavelet domain include a cross-wavelet transform.

6. The computer-implemented method of claim 2, wherein mathematical characteristics of the non-linear domain include cross-entropy.

7. The computer-implemented method of claim 2, wherein mathematical characteristics of the phase domain include a Hilbert transform.

8. The computer-implemented method of claim 2, wherein mathematical characteristics of the information domain include at least one of Granger causality and mutual information.

9. The computer-implemented method of claim 1, wherein said specified ranges of time lags and frequency bands are determined empirically from a database of time series representations collected during periods of health and early stages of illness.

10. The computer-implemented method of claim 1, wherein said specified time window has a length determined in dependence on dynamics of a particular disease.

11. The computer-implemented method of claim 1, wherein rates of change are associated with particular diseases.

12. The computer-implemented method of claim 1, wherein identifying a rank order comprises identification of which time series and physiological processes are most related to each other to identify patterns of entrainments.

13. The computer-implemented method of claim 1, wherein identifying the rank order of the cross-measures with regard to their expected distributions comprises identification of how extreme the measures and cross-measures are compared to a large database of observed values.

14. The computer-implemented method of claim 1, wherein determining the concordance or discordance among time series representations of physiological processes comprises determining whether the entrainment leads to simultaneous or lagged joint increases and decreases (concordant), or to opposite changes in the values of one time series with respect to the other (discordant).

15. A computer-implemented method for advance detection of sub-acute, potentially catastrophic illness in a patient from abnormal entrainment of multidimensional time series representations of physiological processes of the patient, comprising:
defining illness signatures using parameters of entrainment among time series representations of physiological processes in clinically annotated databases;
mathematically characterizing m time series of said patient by m×n parameters selected from said defined illness signatures;
calculating at least one probability of a specific illness (p(illness)) using a predictive mathematical or statistical model that uses the m×n parameters; and
displaying said p(illness) on a display device.

16. A system for advance detection of sub-acute, potentially catastrophic illness in a patient from abnormal entrainment of multidimensional time series representations of physiological processes of the patient, comprising:
a processor configured to:
calculate mathematical characteristics of relationships between a plurality of simultaneous time series representations of physiological processes of said patient in a plurality of domains;
determine cross-measures of said time series representations in specified ranges of time lags and frequency bands as functions of at least two simultaneous time series representations of physiological processes;
calculate rates of change of said cross-measures over a specified time window of predefined length;
identify a rank order of said cross-measures with respect to each other;
identify a rank order of said cross-measures with respect to their expected distributions;
determine the concordance or discordance among said time series representations;
determine the percentile or rank of the determined cross-measures with respect to a database of cross-measures;
calculate a probability of an illness from a predefined multivariable statistical model that employs at least some observed cross-measure parameters and/or their percentile or rank; and
a display device configured to display said probability of illness.

17. The system of claim 16, wherein said plurality of time domains in said calculating mathematical characteristics includes at least two of: time domain, frequency domain, wavelet domain, non-linear domain, phase domain, and information domain.

18. The system of claim 17, wherein mathematical characteristics of the time domain include at least one of autocorrelation, cross-correlation and covariance.

19. The system of claim 17, wherein mathematical characteristics of the frequency domain include at least one of frequency spectra using a Fourier transform, Lomb periodogram, cross-spectra, coherence, or transfer functions.

20. The system of claim 17, wherein mathematical characteristics of the wavelet domain include a cross-wavelet transform.

21. The system of claim 17, wherein mathematical characteristics of the non-linear domain include cross-entropy.

22. The system of claim 17, wherein mathematical characteristics of the phase domain include a Hilbert transform.

23. The system of claim 17, wherein mathematical characteristics of the information domain include at least one of Granger causality and mutual information.

24. The system of claim 16, wherein said specified ranges of time lags and frequency bands are determined empirically from a database of time series representations collected during periods of health and early stages of illness.

25. The system of claim 16, wherein said specified time window has a length determined in dependence on dynamics of a particular disease.

26. The system of claim 16, wherein rates of change are associated with particular diseases.

27. The system of claim 16, wherein identifying a rank order comprises identification of which time series and physiological processes are most related to each other to identify patterns of entrainments.

28. The system of claim 16, wherein identifying the rank order of the cross-measures with regard to their expected distributions comprises identification of how extreme the measures and cross-measures are compared to a large database of observed values.

29. The system of claim 16, wherein determining the concordance or discordance among time series representations of physiological processes comprises determining whether the entrainment leads to simultaneous or lagged joint increases and decreases (concordant), or to opposite changes in the values of one time series with respect to the other (discordant).

30. A system for advance detection of sub-acute, potentially catastrophic illness in a patient from abnormal entrainment of multidimensional time series representations of physiological processes of the patient, comprising:
a processor configured to:
define illness signatures using parameters of entrainment among time series representations of physiological processes in clinically annotated databases;
mathematically characterize m time series of said patient by m×n parameters selected from said defined illness signatures;
calculate at least one probability of a specific illness (p(illness)) using a predictive mathematical or statistical model that uses the m×n parameters; and
a display configured to display said p(illness).
